(19) United States
(12) Patent Application Publication
(10) Pub. No.: US 2008/0307537 A1
(43) Pub. Date: Dec. 11, 2008

(54) Compositions and Methods for the Identification, Assessment, Prevention, and Therapy of Neurological Diseases, Disorders and Conditions

(75) Inventor: Robert M. Bachoo, Roslindale, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: DANA-FARBER CANCER INSTITUTE, INC., BOSTON, MA (US)

(21) Appl. No.: 11/910,144

(22) PCT Filed: Mar. 21, 2006
(86) PCT No.: PCT/US2006/011960

§ 371 Date: Jun. 17, 2008

Related U.S. Application Data
(60) Provisional application No. 60/667,922, filed on Mar. 31, 2005.

(51) Int. Cl. A01K 67/027; C12Q 1/68; G01N 33/53; C12N 15/63; C12N 5/00

(52) U.S. Cl. 800/18; 435/6; 435/7.1; 435/320.1; 435/325

(57) ABSTRACT

A material comprising a plurality of closed cells is provided, the space within each cell being substantially evacuated. This may be achieved by sealing a dimpled film to a sealing film in a vacuum so that each dimple is closed while under vacuum to form an evacuated closed cell.

### CROSS-REFERENCE TO RELATED APPLICATIONS

This application claims the benefit of priority to U.S. Provisional Application No. 60/667,922, filed on Mar. 31, 2005, the contents of which are hereby incorporated in their entirety.

### GOVERNMENT FUNDING

Work described herein was supported, at least in part, by the National Institutes of Health (NIH) under grant numbers 5KO8NS42737, 5K08CA82241, HD007466, P01CA95616, 1R01HG02341, and P2OCA96470. The government may have certain rights to this invention.

### BACKGROUND OF THE INVENTION

The vertebrate central nervous system (CNS) is comprised of three predominant cell types—neurons, oligodendrocytes and astrocytes—that arise from multipotent neural stem cells (NSCs). Recent studies have provided considerable insight into the development and diversity of neurons and the oligodendrocyte lineage (Shirasaki, R. & Pfaff, S. L. (2002) Annu Rev Neurosci 25, 251-81; Miller, R. H. (2002) Prog Neurobiol 67, 451-67). In contrast, there is a more limited molecular understanding of the development and diversity of the astrocyte lineage.

Historically, astrocytes have been viewed as a homogenous population of cells functioning to provide passive support by supply of essential substrates and removing toxic metabolites. This perceived limited functional range of the astrocyte is not consistent with the emerging data that these cells may retain stem cell like properties (Steindler, D. A. & Laywell, E. D. (2003) Glia 43, 62-9; Doetsch, F. (2003) Nat Neurosci 6, 1127-1134) and modulate almost every facet of functional neural networks (Fields, R. D. & Stevens-Graham, B. (2002) Science 298, 556-62; Newman, E. A. (2003) Trends Neurosci 26, 536-42). For instance, astrocytes may express voltage gated ion channels and neurotransmitter receptors that are co-activated at synapses and then participate in removing potentially toxic excitatory amino acids from synapses by high affinity transporters (Auld, D. S. & Robitaille, R. (2003) Neuron 40, 389-400). Astrocyte involvement in neuron homeostasis may also extend to trophic support (Song, H., Stevens, C. F. & Gage, F. H. (2002) Nature 417, 39-44), antioxidant functions, and production of critical substrates for neuron membrane synthesis. Dysregulation of these and other putative astrocyte functions have been variously implicated in the pathogenesis of numerous developmental, genetic, idiopathic and acquired neurodegenerative diseases (Nedergaard, M., et al. (2003) Trends Neurosci 26, 523-30).

To date, precise genetic analyses of the astrocyte in normal physiology and disease processes have been limited to in vitro studies utilizing specific glial differentiation model systems (Liu, Y., et al. (2002) Glia 40, 25-43; De Smet, C., et al. (2002) J Neurochem 81, 575-88; Geschwind, D. H., et al. (2001) Neuron 29, 325-39). These important efforts have focused on specialized aspects of early glial differentiation and as such have yielded limited information on the diverse roles of astrocytes in normal brain. However, the challenge remains to develop a comprehensive molecular profile of the astrocyte lineage that reflects its apparent developmental complexity, its full range of physiological capacities, its lineage heterogeneity as well as its role in the pathogenesis of numerous developmental, genetic, idiopathic and acquired neurological diseases, disorders, or conditions.

### SUMMARY OF THE INVENTION

The present invention is based, at least in part, on the identification of correlations between certain markers, e.g., nucleic acid markers and protein markers, involved in neural cell survival and neural cell homeostasis, e.g., markers differentially expressed in astrocytes, and in subjects suffering from neurological diseases, disorders, or conditions. The invention relates to compositions, kits, and methods for detecting, characterizing, preventing, and treating human neurological diseases, disorders, or conditions.

Accordingly, one aspect of the invention pertains to a method of assessing whether a subject is afflicted with a neurological disease, disorder or condition, the method comprising comparing: a) the amount and/or activity of at least one marker in a subject sample, wherein the at least one marker is selected from the group consisting of the markers listed in Table 2, and b) the normal amount and/or activity of at the least one marker in a control sample from a subject not afflicted with a neurological disease, disorder, or condition, wherein modulation of the amount and/or activity of the at least one marker in the subject sample compared to the normal amount and/or activity is an indication that the subject is afflicted with a neurological disease, disorder or condition. In one embodiment, the amount of at least one marker is compared. In another embodiment, the activity of at least one marker is compared. In yet another embodiment, the amount of at least one marker is determined by determining the level of expression of a marker. In another embodiment, the amount of at least one marker is determined by determining the copy number of the marker. In a further embodiment, the level of expression of the at least one marker is assessed by detecting the presence in the sample of a protein corresponding to the marker. In yet a further embodiment, the presence of the protein is detected using a reagent which specifically binds the protein, e.g., an antibody, an antibody derivative, or an antibody fragment. In one embodiment, the level of expression of the at least one marker in the sample is assessed by detecting the presence of a transcribed polynucleotide, e.g., an mRNA or a cDNA, or portion thereof, wherein the transcribed polynucleotide comprises the marker. In yet another embodiment, the step of detecting further comprises amplifying the transcribed polynucleotide. In one embodiment, the level of expression of the at least one marker in the sample is assessed by detecting the presence in the sample of a transcribed polynucleotide which anneals with the marker or anneals with a portion of a polynucleotide wherein the polynucleotide comprises the at least one marker, under stringent hybridization conditions. In one embodiment, the subject sample is selected from the group consisting of neuroglial tissue, whole blood, serum, plasma, buccal scrape, saliva, cerebrospinal fluid, spinal fluid, urine and stool. In another embodiment, the at least one marker is selected from the subset of markers in listed in Table 5 or Table 7.

Another aspect of the invention pertains to a method of assessing the efficacy of a test compound for treating or preventing a neurological disease, disorder or condition in a subject. The method comprises comparing: a) the amount and/or activity of at least one marker in a first sample obtained from the subject and maintained in the presence of the test compound, wherein the marker is selected from the group consisting of the markers listed in Table 2, and b) the amount and/or activity of the at least one marker in a second sample obtained from the subject and maintained in the absence of the test compound, wherein a modulation of the amount and/or activity of the at least one marker in the first sample from Table 2, as compared to the second sample, is an indication that the test compound is efficacious for treating or preventing a neurological disease, disorder or condition in the subject. In one embodiment, the first and second samples are portions of a single sample obtained from the subject. In another embodiment, the first and second samples are portions of pooled samples obtained from the subject. In one embodiment, the at least one marker is selected from the subset of markers listed in Table 5 or Table 7.

Another aspect of the invention features a method of assessing the efficacy of a therapy for treating or preventing a neurological disease, disorder or condition in a subject. The method comprises comparing: a) the amount and/or activity of at least one marker in a first sample obtained from the subject prior to administering at least a part of the therapy to the subject, wherein the marker is selected from the group consisting of the markers listed in Table 2, and b) the amount and/or activity of the at least one marker in a second sample obtained from the subject following the administration of at least a part of the therapy, wherein modulation of the amount and/or activity of the at least one marker in the first sample, as compared to the second sample, is an indication that the therapy is efficacious for treating or preventing a neurological disease, disorder or condition in the subject. In one embodiment, the at least one marker is selected from the subset of markers listed in Table 5 or Table 7.

In yet another aspect, the invention features a method of selecting a composition capable of modulating a symptom of a neurological disease, disorder or condition. The method comprises: a) providing a sample comprising an astrocyte; b) contacting said sample with a test compound; and c) determining the ability of the test compound to modulate the amount and/or activity of at least one marker, wherein the marker is selected from the group consisting of the markers listed in Table 2; thereby identifying a composition capable of modulating a symptom of a neurological disease, disorder or condition. In one embodiment, the astrocytes are isolated from an animal model of a neurological disease, disorder or condition. In another embodiment, the astrocytes are isolated from a neural cell line. In yet another embodiment, the astrocytes are isolated from a subject suffering from a neurological disease, disorder or condition. In one embodiment, the at least one marker is selected from the subset of markers listed in Table 5 or Table 7. In another embodiment, the method further comprises administering the test compound to an animal model of a neurological disease, disorder or condition.

In another aspect, the invention features a method of treating a subject afflicted with a neurological disease, disorder or condition. The method comprises administering to the subject a therapeutically effective amount of a compound which modulates the amount and/or activity of a gene or protein corresponding to at least one marker listed in Table 2, thereby treating a subject afflicted with a neurological disease, disorder or condition. Another aspect of the invention features a method for modulating neural homeostasis in a subject comprising administering to the subject a compound which modulates the amount and/or activity of a gene or protein corresponding to at least one marker listed in Table 2, thereby modulating neural homeostasis in a subject. Yet another aspect of the invention features a method of modulating neural cell survival in a subject comprising administering to the subject a compound which modulates the amount and/or activity of a gene or protein corresponding to at least one marker listed in Table 2, thereby modulating neural cell survival in said subject. In one embodiment, the compound is administered in a pharmaceutically acceptable formulation. In another embodiment, the compound is an antibody, an antibody derivative, or an antigen binding fragment thereof, which specifically binds to a protein corresponding to said marker. In a further embodiment, the antibody, antibody derivative, or antigen binding portion thereof, is conjugated to a toxin or a chemotherapeutic agent. In one embodiment, the compound is an RNA interfering agent, e.g., an siRNA or an shRNA molecule, which inhibits expression of a gene corresponding to said marker. In another embodiment, the compound is an antisense oligonucleotide complementary to a gene corresponding to said marker. In yet another embodiment, the compound is a peptide or peptidomimetic. In one embodiment, the compound is a small molecule which inhibits activity of said marker. In a further embodiment, the small molecule inhibits a protein-protein interaction between a marker and a target protein. In one embodiment, the compound is an aptamer which inhibits expression or activity of said marker. In another embodiment, the at least one marker is selected from the subset of markers listed in Table 5 or Table 7.

Another aspect of the invention features a kit for assessing the suitability of each of a plurality of compounds for treating or preventing a neurological disease, disorder or condition in a subject. The kit comprises: a) the plurality of compounds; and b) a reagent for assessing the amount and/or activity of at least one marker selected from the group consisting of the markers listed in Table 2. Yet another aspect of the invention features a kit for assessing whether a subject is afflicted with a neurological disease, disorder or condition. The kit comprises reagents for assessing the amount and/or activity of at least one marker selected from the group consisting of the markers listed in Table 2. In one embodiment, the at least one marker is selected from the subset of markers listed in Table 5 or Table 7.

One aspect of the invention features a method of making an isolated hybridoma which produces an antibody useful for assessing whether a subject is afflicted with a neurological disease, disorder or condition. The method comprises: isolating a protein corresponding to a marker selected from the group consisting of the markers listed in Table 2; immunizing a mammal using the isolated protein; isolating splenocytes from the immunized mammal; fusing the isolated splenocytes with an immortalized cell line to form hybridomas; and screening individual hybridomas for production of an antibody which specifically binds with the protein to isolate the hybridoma. Another aspect of the invention features an antibody produced by a hybridoma made by the foregoing method. In one embodiment, the at least one marker is selected from the subset of markers listed in Table 5 or Table 7.

In another aspect, the invention features a kit for assessing the presence in a sample of cells afflicted with a neurological disease, disorder or condition. The kit comprises an antibody, an antibody derivative, or fragment thereof, wherein the antibody, antibody derivative, or fragment thereof specifically binds with a protein corresponding to a marker selected from the group consisting of the markers listed in Table 2. Another aspect of the invention features a kit for assessing the presence in a sample of cells afflicted with a neurological disease, disorder or condition, the kit comprising a nucleic acid probe wherein the probe specifically binds with a transcribed polynucleotide, e.g., mRNA or cDNA, corresponding to a marker selected from the group consisting of the markers listed in Table 2. In one embodiment, the nucleic acid probe is a molecular beacon probe. In another embodiment, the at least one marker is selected from the subset of markers listed in Table 5 or Table 7.

Yet another aspect of the invention features a recombinant vector comprising an astrocyte-specific promoter operably linked to a Cre recombinase. In one embodiment, the vector further comprises an inducible fusion protein. In one embodiment, the inducible fusion protein comprises the estrogen receptor (ERT2). In another embodiment, the inducible fusion protein is induced by tamoxifen.

Another aspect of the invention features a cell or cell line comprising the recombinant vectors of the invention. Yet another aspect of the invention features a non-human animal containing the recombinant vectors of the invention.

One aspect of the invention features a recombinant vector comprising an astrocyte-specific promoter operably linked to sites of inducible recombination that flank a reporter sequence. In one embodiment, the reporter sequence comprises LacZ. In another embodiment, the reporter sequence comprises GFP. In yet another embodiment, the reporter sequence comprises EGFP. In a further embodiment, the sites of inducible recombination are lox sites. In yet a further embodiment, the sites of inducible recombination are loxP sites.

Another aspect of the invention features a recombinant vector comprising a astrocyte-specific promoter operably linked to an inducible fusion protein, and operably linked to a nucleotide sequence containing at least one exon of the EGFR gene.

Yet another aspect of the invention features a method of identifying the presence of astrocytes in a cell sample comprising determining the amount and/or activity of at least one marker in Table 2, to thereby identify the presence of astrocytes in the cell sample. In one embodiment, the at least one marker is selected from the subset of markers listed in Table 5 or Table 7.

### BRIEF DESCRIPTION OF THE DRAWINGS

FIG. 1 depicts the experimental strategy to identify the astrocyte transcriptome. Embryonic neural stem cells (NSCs) were differentiated into astrocytes by exposure to either serum (from 24 hours to 5 days), or CNTF, BMP2, or PACAP for 5 days. Primary cortical astrocyte cultures were isolated from postnatal mice (P1-2). Pure neuronal cultures were derived from embryonic E13.5 hippocampus. In addition, the gray matter, corpus callosum and glial limitans were microdissected from coronal sections of the telencephalon of postnatal (P0, P2, P5, P10) and adult mice. RNA was isolated from all of the above samples and was hybridized to the Affymetrix U74 oligonucleotide micro-arrays. The arrays were analyzed by D-chip software. Differentially expressed genes were analyzed by (i) unsupervised hierarchical clustering, (ii) R-SVM and (ii) threshold criteria. Genes differentially expressed by neurons (>3LBFC) were subtracted from the data. Candidate astrocyte genes were validated by RNA in situ hybridization (ISH) combined with immunohistochemistry (IHC). Finally, a novel clustering algorithm was used to identify additional astrocyte specific genes that ‘tightly cluster’ with the validated astrocyte genes

FIGS. 2A-2B depict the identification of astrocyte-specific candidate genes by UHC and R-SVM. (A) UHC analysis divided the experimental samples into two distinct groups that cluster on separate branches of the dendrogram. With all of the astrocyte samples clustered together, the short vertical distance between the astrocyte samples in the dendrogram indicated statistical similarity between the cortical astrocyte samples and the various differentiated astrocytes. Similarly, the CC, WM and GL cluster together suggesting a common transcriptional signal. The NSC, the embryonic (E13.5) cortex and neuronal lineage committed cells clustered together. The expression level matrix is shown representing standardized values from −3 (light gray, below the mean) to 3 (dark gray, above the mean). The mean (0 value) is represented by the white color. Rows correspond to different genes, and the columns represent the various experimental samples. When all in vitro and in vivo experimental samples were used, UHC generated a large cluster of 393 genes, which are strongly expressed among the in vitro astrocyte samples. Although GFAP is among this group of astrocyte-associated genes, there is no obvious GFAP sub cluster. (B) R-SVM, a novel class prediction tool, identified a subset of 85 genes, which contribute most to distinguishing astrocytes from undifferentiated or early lineage committed cells. The majority (53%) of the astrocyte candidate genes were from only in vitro astrocyte experimental samples, the remainder were differentially expressed both in cultured astrocytes and among the brain subregions. Regions of overlap indicate genes which were differentially expressed in both experimental samples.

FIGS. 3A-3C depict astrocytic candidate gene validation. (A). Candidate astrocyte genes with ‘glial’ expression based on similarity to the reference gene expression patterns for GFAP ISH and/or GFAP IHC were chosen for further validation. Note marked abundance of GFAP RNA in glia limitans (gl) and corpus callosum (cc) (arrowheads) and relative absence in cortical gray matter (cx). (B). The majority of validated genes showed a broad ‘pan-astrocytic’ pattern of expression in gray and white matter astrocytes (shown here, Clusterin (Clu), Apolipoprotein E (ApoE), Glutathione S-Transferase (GSTm), Aldolase 3 (Aldo3), and Cystatin 3 (Cst3); a subset of each which were GFAP positive. (C.) Several validated astrocyte genes showed a restricted expression pattern in subsets of astrocytes. Phospholipase A, group7 (Pla2g7) was predominantly expressed in cortical gray matter astrocytes while Aquaporin 4 (Aqp4) was highly abundant in glial limitans regions.

FIGS. 4A-4B depict tight cluster analysis and validated astrocyte specific genes which identifies additional astrocyte candidate genes. (A) Tight cluster analysis identified 4 tight clusters (shown in descending order of tightness, upper left) by inclusion of a total of 6 validated astrocyte genes across both astrocytes in cell culture and among the brain subregions but not in NSCs, neurons or embryonic brain. Two of these tight clusters are enlarged and shown with gene names, (top and bottom clusters have 28 and 51 genes, respectively). Validated genes are shown, top cluster 1 gene; bottom cluster 3 genes. (B) Similar tight cluster analysis using only the cell culture samples yield 9 clusters (shown on the left in descending order of tightness) identified by 16 in situ validated genes, the 3 enlarged clusters, with a total of 12, 26 and 40 genes, have 2, 2, and 6 validated astrocyte genes, respectively.

FIG. 5 illustrates that replicate samples within a given experimental modality or tissue type demonstrated a high degree of reproducibility (correlation coefficient 0.95-0.99) and when analyzed as groups, highlight the distinctiveness of the astrocyte profile from the profiles of neurons, NSCs and embryonic cortex.

FIGS. 6A-6F are graphs depicting the expression of astrocyte-specific genes in the brain and major organs from E13.5, P0, P5 and adult mice assessed by quantitative PCR.

### DETAILED DESCRIPTION OF THE INVENTION

The present invention is based, at least in part, on the molecular characterization of neuroglia, e.g., astrocytes. In particular, the present invention is based on the characterization of the astrocyte on the molecular level through transcriptional analysis of distinct astrocyte-rich cultures and CNS tissues using various bioinformatic approaches, as well as a validation scheme, to reveal distinctive patterns of expression.

The present invention provides newly discovered correlations between certain astrocyte-specific markers (e.g., nucleic acid markers and protein markers which are differentially expressed in astrocytes as compared to other brain cells), and neurological diseases, disorders, and conditions. Accordingly, methods are provided herein for utilizing the markers of the invention for detecting the presence of a neurological disease, disorder, or condition in a sample, the absence of a neurological disease, disorder or condition in a sample, and other characteristics of a neurological disease, disorder or condition that are relevant to prevention, diagnosis, characterization, and therapy of a neurological disease, disorder or condition in a subject.

In one embodiment, certain markers of the invention correlate with the grade of tumor, e.g., glioma, tumor prognosis, and treatment response of a tumor. Therefore, the present invention provides methods for diagnosing tumor grade, e.g., glioma grade, clinical outcome, and prognosis for a subject afflicted with a tumor, e.g., a glioma. For example, the markers of the present invention may be used to determine whether a tumor, e.g., a glioma, is a high grade tumor or a low grade tumor, to predict the responsiveness of a tumor to certain treatment regimens, and to determine the prognosis of a subject with a tumor, e.g., a glioma.

In another embodiment of the invention, nucleic acid molecules are provided which are useful for the construction of transgenic models of neurological diseases, disorders, and conditions, including animal models for cancer, e.g., brain tumor, e.g., glioma animal models.

Various aspects of the invention are described in further detail in the following subsections:

### I. DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

As used herein, a “neuron” or “neural cell” is a cell that has two processes, e.g., axons and dendrites, and is capable of generating an action potential. Neurons have synapses that release and use neurotransmitters.

As used herein, “neuroglia” refers to the non-neuronal cellular elements of the central and peripheral nervous systems that have a resting potential. Neuroglia were formerly believed to be merely supporting cells but are now known to have important metabolic functions, since they are invariably interposed between neurons and the blood vessels supplying the nervous system. In central nervous tissue, neuroglia include astrocytes, oligodendroglia cells, ependymal cells, and microglia cells. The satellite cells of ganglia and the neurolemmal or Schwann cells around peripheral nerve fibers are the oligodendroglia cells of the peripheral nervous system.

As used herein, an “astrocyte” is a neuroglial cell which has a characteristic star-like shape and retains characteristics of neural stem cells (NSCs). An astrocyte is of ectodermal origin, and is characterized by fibrous, protoplasmic, or plasmatofibrous processes. Astrocytes provide physical and nutritional support for neurons, e.g., “neural homeostasis” and “neural cell survival” and, as such, play a modulatory role in various neurological diseases, disorders, and conditions. For example, an astrocyte is capable of performing one or more of the following functions which are necessary for neural cell survival and/or neural homeostasis: 1) removing brain debris; 2) transporting nutrients to neurons; 3) holding neurons in place; 4) digesting portions of dead neurons; 5) modulating neurotransmitter release; 6) producing substrates for neuron membrane synthesis; and 7) regulating the content of extracellular space, e.g., removing neurotransmitters.

As such, if any of these activities are disrupted, a neurological disease, disorder, or condition will develop. The term “neurological disease, disorder or condition” e.g., diseases, disorders and conditions of the central nervous system (CNS), is intended to be used in its broadest sense to include diseases, disorders or conditions, such as cognitive and neurodegenerative disorders, pain, and cancer or tumors of the central nervous system. Non-limiting examples of cognitive and neurodegenerative disorders include Alzheimer's disease, dementias related to Alzheimer's disease (such as Pick's disease), Parkinson's and other Lewy diffuse body diseases, senile dementia, Huntington's disease, Gilles de la Tourette's syndrome, musculoskeletal diseases, multiple sclerosis, amyotrophic lateral sclerosis, progressive supranuclear palsy, epilepsy, and Jakob-Creutzfieldt disease; autonomic function disorders such as hypertension and sleep disorders, and neuropsychiatric disorders, such as depression, schizophrenia, schizoaffective disorder, korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), and bipolar affective neurological disorders, e.g., migraine and obesity.

The term “pain” is defined herein based on the recommendation of International Association for the Study of Pain, as an unpleasant sensory and emotional experience associated with actual or potential tissue damage, or described in terms of such damage. Pain can be classified to include transient, acute and chronic pain. Acute and chronic pain are further categorized based on organ or tissue localization, whether it is malignant, e.g., having a cancerous origin, or nonmalignant. Furthermore, pain may be characterized as nociceptive, neuropathic or a combination thereof.

Non-limiting examples of pain that are contemplated by the invention include posttherapeutic neuralgia, posttherapeutic neuralgia, diabetic neuropathy, postmastectomy pain syndrome, stump pain, reflex sympathetic dystrophy, trigeminal neuralgia, neuropathic pain, orofacial neuropathic pain, diabetic neuropathy, causalgia, phantom limb pain, osteoarthritis, rheumatoid arthritis, pain associated with cancer, pain associated with HIV, fibromyalgia syndrome, tension myalgia, Guillian-Barre syndrome, Meralgia paraesthetica, burning mouth syndrome, fibrocitis, myofascial pain syndrome, idiopathic pain disorder, temporomandibular joint syndrome, atypical odontalgia, loin pain, haematuria syndrome, non-cardiac chest pain, low back pain, chronic nonspecific pain, psychogenic pain, musculoskeletal pain disorder, chronic pelvic pain, nonorganic chronic headache, tension-type headache, cluster headache, migraine and other conditions associated with chronic cephalic pain, complex regional pain syndrome, vaginismus, nerve trunk pain, somatoform pain disorder, cyclical mastalgia, chronic fatigue syndrome, multiple somatization syndrome, chronic pain disorder, somatization disorder, tabes dorsalis, spinal cord injury, central pain, posttherapeutic pain, noncardiac chest pain, irritable bowel syndrome, central post-stroke pain, Syndrome X, facial pain, idiopathic pain disorder, posttraumatic rheumatic pain modulation disorder (fibrositis syndrome), hyperalgesia, inflammatory pain and Tangier disease.

Non-limiting examples of cancers of the central nervous system include gliomas. As used herein, a “glioma” is a tumor of the central nervous system that develops from neuroglial cells and can develop as a primary brain tumor or a primary spinal cord tumor. Within the brain, gliomas usually occur in the cerebral hemispheres but may also affect other areas, especially the optic nerve, the brain stem and, particularly among children, the cerebellum. Gliomas are classified into several groups, such as, for example, astrocytomas, well-differentiated astrocytomas, anaplastic astrocytomas, and Glioblastoma Multiforme. Furthermore, under the current World Health Organization (WHO) grading system, gliomas are graded (I to IV) on the basis of a proliferative index and the presence or absence of neovascular proliferation.

Additional neurological diseases, disorders and conditions also contemplated by the present invention include ischemic disease, diabetic neuropathy, anti-cancer-agent-intoxicated neuropathy, retinal pigment degeneration, glaucoma, an anoxic episode, an injury to the brain and other parts of the CNS caused by trauma or other injury, a blow to the head, a spinal injury, a thromboembolic or hemorrhagic stroke, a cerebral vasospasm, hypoglycemia, cardiac arrest, cerebral ischemia or cerebral infarction, ischemic, hypoxic or anoxic brain damage, spinal cord injury, tissue ischemia and reperfusion injury. Further CNS-related disorders include, for example, those listed in the American Psychiatric Association's Diagnostic and Statistical manual of Mental Disorders (DSM), the most current version of which is incorporated herein by reference in its entirety.

A “marker”, e.g., an astrocyte-specific marker, e.g., a marker which is differentially expressed in astrocytes as compared to other brain cells, is a gene or protein that may be altered, wherein said alteration is associated with a neurological disease, disorder or condition, neural cell survival and/or neural cell homeostasis. The alteration may be in amount, structure, and/or activity in a neuroglial tissue or cell, e.g., an astrocyte, as compared to its amount, structure, and/or activity, in a normal or healthy tissue or cell (e.g., a control), and is associated with a disease state, such as a neurological disease, disorder or condition. For example, a marker of the invention which is associated with a neurological disease, disorder or condition may have altered copy number, expression level, protein level, protein activity, or methylation status, in a neuroglial tissue or cell as compared to a normal, healthy tissue or cell. Furthermore, a “marker” includes a molecule whose structure is altered, e.g., mutated (contains an allelic variant), e.g., differs from the wild type sequence at the nucleotide or amino acid level, e.g., by substitution, deletion, or addition, when present in a tissue or cell associated with a disease state, such as a neurological disease, disorder or condition.

The term “altered amount” or “modulated amount”, used interchangeably herein, of a marker, or “altered level” or “modulated level”, used interchangeably herein, of a marker refers to a modulated, e.g., increased or decreased, copy number of a marker or chromosomal region, and/or modulated, e.g., increased or decreased, expression level of a particular marker gene or genes in a neurological disease, disorder or condition sample, as compared to the expression level or copy number of the marker in a control sample. The term “altered amount” or “modulated amount” of a marker also includes a modulated, e.g., an increased or decreased, protein level of a marker in a sample, e.g., a neurological disease, disorder or condition sample, as compared to the protein level of the marker in a normal, control sample. Furthermore, an altered or modulated amount of a marker may be determined by detecting the methylation status of a marker, as described herein, which may affect the expression or activity of a marker.

The amount of a marker, e.g., expression or copy number of a marker, or protein level of a marker, in a subject is “significantly” higher or lower than the normal amount of a marker, if the amount of the marker is greater or less, respectively, than the normal level by an amount greater than the standard error of the assay employed to assess amount, and preferably at least twice, and more preferably three, four, five, ten or more times that amount. Alternately, the amount of the marker in the subject can be considered “significantly” higher or lower than the normal amount if the amount is at least about two, and preferably at least about three, four, or five times, higher or lower, respectively, than the normal amount of the marker.

The “copy number of a gene” or the “copy number of a marker” refers to the number of DNA sequences in a cell encoding a particular gene product. Generally, for a given gene, a mammal has two copies of each gene. The copy number can be increased, however, by gene amplification or duplication, or reduced by deletion.

The “normal” copy number of a marker or “normal” level of expression of a marker is the level of expression, copy number of the marker, in a biological sample, e.g., a sample containing tissue or cells, e.g., neuroglial tissue or cells, e.g., astrocytes, whole blood, serum, plasma, buccal scrape, saliva, spinal fluid, cerebrospinal fluid, urine, stool, from a subject, e.g. a human, not afflicted with a neurological disease, disorder or condition, e.g., a control sample.

The term “altered level of expression” used interchangeably herein with “modulated level of expression” of a marker refers to an expression level or copy number of a marker in a test sample e.g., a sample derived from a patient suffering from a neurological disease, disorder or condition, that is modulated, e.g., greater or less, than the standard error of the assay employed to assess expression or copy number, and is preferably at least twice, and more preferably three, four, five or ten or more times the expression level or copy number of the marker in a control sample (e.g., sample from a healthy subjects not having the associated neurological disease, disorder or condition) and preferably, the average expression level or copy number of the marker in several control samples. The altered level of expression is modulated, e.g., greater or less, than the standard error of the assay employed to assess expression or copy number, and is preferably at least twice, and more preferably three, four, five or ten or more times the expression level or copy number of the marker in a control sample (e.g., sample from a healthy subjects not having the associated a neurological disease, disorder or condition) and preferably, the average expression level or copy number of the marker in several control samples.

An “overexpression” or “significantly higher level of expression or copy number” of a marker refers to an expression level or copy number in a test sample that is greater than the standard error of the assay employed to assess expression or copy number, and is preferably at least twice, and more preferably three, four, five or ten or more times the expression level or copy number of the marker in a control sample (e.g., sample from a healthy subject not afflicted with a neurological disease, disorder or condition) and preferably, the average expression level or copy number of the marker in several control samples.

An “underexpression” or “significantly lower level of expression or copy number” of a marker refers to an expression level or copy number in a test sample that is greater than the standard error of the assay employed to assess expression or copy number, but is preferably at least twice, and more preferably three, four, five or ten or more times less than the expression level or copy number of the marker in a control sample (e.g., sample from a healthy subject not afflicted with a neurological disease, disorder or condition) and preferably, the average expression level or copy number of the marker in several control samples

“Methylation status” of a marker refers to the methylation pattern, e.g., methylation of the promoter of the marker, and/or methylation levels of the marker. DNA methylation is a heritable, reversible and epigenetic change. Yet, DNA methylation has the potential to alter gene expression, which has developmental and genetic consequences. DNA methylation has been linked to cancer, as described in, for example, Laird, et al. (1994) Human Molecular Genetics 3:1487-1495 and Laird, P. (2003) Nature 3:253-266, the contents of which are incorporated herein by reference. For example, methylation of CpG oligonucleotides in the promoters of tumor suppressor genes can lead to their inactivation. In addition, alterations in the normal methylation process are associated with genomic instability (Lengauer, et al. Proc. Natl. Acad. Sci. USA 94:2545-2550, 1997). Such abnormal epigenetic changes may be found in many types of cancer, e.g., gliomas, and can, therefore, serve as potential markers for oncogenic transformation. For example, see Costell, J. F. (2003) Front. Biosci. 8:s175-184.

Methods for determining methylation include restriction landmark genomic scanning (Kawai, et al., Mol. Cell. Biol. 14:7421-7427, 1994), methylation-sensitive arbitrarily primed PCR (Gonzalgo, et al., Cancer Res. 57:594-599, 1997); digestion of genomic DNA with methylation-sensitive restriction enzymes followed by Southern analysis of the regions of interest (digestion-Southern method); PCR-based process that involves digestion of genomic DNA with methylation-sensitive restriction enzymes prior to PCR amplification (Singer-Sam, et al., Nucl. Acids Res. 18:687, 1990); genomic sequencing using bisulfite treatment (Frommer, et al., Proc. Natl. Acad. Sci. USA 89:1827-1831, 1992); methylation-specific PCR (MSP) (Herman, et al. Proc. Natl. Acad. Sci. USA 93:9821-9826, 1992); and restriction enzyme digestion of PCR products amplified from bisulfite-converted DNA (Sadri and Hornsby Nucl. Acids Res. 24:5058-5059, 1996; and Xiong and Laird Nucl. Acids. Res. 25:2532-2534, 1997); PCR techniques for detection of gene mutations (Kuppuswamy, et al., Proc. Natl. Acad. Sci. USA 88:1143-1147, 1991) and quantitation of allelic-specific expression (Szabo and Mann Genes Dev. 9:3097-3108, 1995; and Singer-Sam, et al., PCR Methods Appl. 1: 160-163, 1992); and methods described in U.S. Pat. No. 6,251,594, the contents of which are incorporated herein by reference. An integrated genomic and epigenomic analysis as described in Zardo, et al. (2000) Nature Genetics 32:453-458, may also be used.

The term “altered activity” used interchangeably herein with “modulated activity” of a marker refers to an activity of a marker which is modulated, e.g., increased or decreased, in a disease state, e.g., in a neurological disease, disorder or condition sample, as compared to the activity of the marker in a normal, control sample. Altered or modulated activity of a marker may be the result of, for example, altered or modulated expression of the marker, altered or modulated protein level of the marker, altered or modulated structure of the marker, or, e.g., an altered or modulated interaction with other proteins involved in the same or different pathway as the marker, or altered or modulated interaction with transcriptional activators or inhibitors, or altered methylation status.

The term “altered structure” used interchangeably herein with “modulated structure” of a marker refers to the presence of mutations or allelic variants within the marker gene or maker protein, e.g., mutations which affect expression or activity of the marker, as compared to the normal or wild-type gene or protein. For example, mutations include, but are not limited to, substitutions, deletions, or addition mutations. Mutations may be present in the coding or non-coding region of the marker.

A “marker nucleic acid” is a nucleic acid (e.g., DNA, mRNA, cDNA) encoded by or corresponding to a marker of the invention. For example, such marker nucleic acid molecules include DNA (e.g., cDNA) comprising the entire or a partial sequence of any of the nucleic acid sequences of the genes set forth in Table 2 or the complement or hybridizing fragment of such a sequence. The marker nucleic acid molecules also include RNA comprising the entire or a partial sequence of any of the nucleic acid sequences of the genes set forth in Table 2 or the complement of such a sequence, wherein all thymidine residues are replaced with uridine residues. A “marker protein” is a protein encoded by or corresponding to a marker of the invention. A marker protein comprises the entire or a partial sequence of a protein encoded by any of the sequences of the genes set forth in Table 2 or a fragment thereof. The terms “protein” and “polypeptide” are used interchangeably herein.

Markers identified herein include diagnostic and therapeutic markers. A single marker may be a diagnostic marker, a therapeutic marker, or both a diagnostic and therapeutic marker.

As used herein, the term “therapeutic marker” includes markers, e.g., markers set forth in Table 2, which are believed to be involved in the development (including maintenance, progression, angiogenesis, and/or metastasis) of a neurological disease, disorder or condition. The neurological disease-, disorder-, or condition-related functions of a therapeutic marker may be confirmed by, e.g., increased or decreased copy number (by, e.g., fluorescence in situ hybridization (FISH) or quantitative PCR (qPCR)) or mutation (e.g., by sequencing), overexpression or underexpression (e.g., by in situ hybridization (ISH), Northern Blot, or qPCR), increased or decreased protein levels (e.g., by immunohistochemistry (IHC)), or increased or decreased protein activity (determined by, for example, modulation of a pathway in which the marker is involved), e.g., in more than about 5%, 6%, 7%, 8%, 9%, 10%, 11%, 12%, 13%, 14%, 15%, 20%, 25%, or more of human neurological diseases, disorders or conditions.

With respect to the functions of a therapeutic marker involved in a cancer, e.g., a tumor (such as a glioma), the function of such a marker may be confirmed by, e.g., (1) the inhibition of neuroglial cell proliferation and growth, e.g., in soft agar, by, e.g., RNA interference (“RNAi”) of the marker; (2) the ability of the marker to enhance transformation of mouse embryo fibroblasts (MEFs) by oncogenes, e.g., Myc and RAS, or by RAS alone; (3) the ability of the marker to enhance or decrease the growth of tumor cell lines, e.g., in soft agar; (4) the ability of the marker to transform primary mouse cells in SCID explant; and/or; (5) the prevention of maintenance or formation of tumors, e.g., tumors arising de novo in an animal or tumors derived from human cancer cell lines, by inhibiting or activating the marker. In one embodiment, a therapeutic marker may be used as a diagnostic marker.

As used herein, the term “diagnostic marker” includes markers, e.g., markers set forth in Table 2, which are useful in the diagnosis of a neurological disease, disorder or condition, e.g., over- or under-activity emergence, expression, growth, remission, recurrence or resistance of a neurological disease, disorder or condition (including a tumor) before, during or after therapy. The predictive functions of the marker may be confirmed by, e.g., (1) increased or decreased copy number (e.g., by FISH or qPCR), overexpression or underexpression (e.g., by ISH, Northern Blot, or qPCR), increased or decreased protein level (e.g., by IHC), or increased or decreased activity (determined by, for example, modulation of a pathway in which the marker is involved), e.g., in more than about 5%, 6%, 7%, 8%, 9%, 10%, 11%, 12%, 13%, 14%, 15%, 20%, 25%, or more of human neurological diseases, disorders or conditions; (2) its presence or absence in a biological sample, e.g., a sample containing tissue or cells, e.g., neuroglial tissue or cells, e.g., astrocytes, whole blood, serum, plasma, buccal scrape, saliva, spinal fluid, cerebrospinal fluid, urine, stool, from a subject, e.g. a human, afflicted with a neurological disease, disorder or condition; or (3) its presence or absence in clinical subset of patients with a neurological disease, disorder or condition (e.g., those responding to a particular therapy or those developing resistance).

A diagnostic marker of the invention includes a marker which is useful for the diagnosis of tumor grade, e.g., glioma grade, tumor prognosis, and treatment response of a tumor. Therefore, the present invention provides methods for diagnosing the grade of tumor (e.g., to determine whether a tumor is a high grade tumor or a low grade tumor), clinical outcome, and prognosis for a subject afflicted with a tumor, e.g., a glioma.

Diagnostic markers also include “surrogate markers,” e.g., markers which are indirect markers of a neurological disease, disorder or condition progression.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic monomers.

As used herein, the term “promoter”, “regulatory sequence”, or “promotor element” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a spatially or temporally restricted manner.

An “RNA interfering agent” as used herein, is defined as any agent which interferes with or inhibits expression of a target gene, e.g., a marker of the invention, by RNA interference (RNAi). Such RNA interfering agents include, but are not limited to, nucleic acid molecules including RNA molecules which are homologous to the target gene, e.g., a marker of the invention, or a fragment thereof, short interfering RNA (siRNA), and small molecules which interfere with or inhibit expression of a target gene by RNA interference (RNAi).

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” or “inhibition of marker gene expression” includes any decrease in expression or protein activity or level of the target gene (e.g., a marker gene of the invention) or protein encoded by the target gene, e.g., a marker protein of the invention. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, may be administered to a patient having or at risk for having of a neurological disease, disorder or condition, to modulate, e.g., inhibit, expression of a marker gene of the invention, e.g., a marker gene which is modulated, e.g., overexpressed, in a neurological disease, disorder or condition (e.g., a marker shown to be increased in a neurological disease, disorder or condition listed in Table 2) and thereby modulate, e.g., treat, prevent, or inhibit, a neurological disease, disorder or condition in the subject.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific promoter”, “spatially-restricted promoter or regulatory sequence”, or “spatially restricted promotor element” is a nucleotide sequence which, when operably linked with a polynucleotide which encodes or specifies a gene product, causes the gene product to be produced in a cell substantially only if the cell is a cell of the tissue type corresponding to the promoter.

A “neuroglial specific promoter or regulatory sequence”” or “neuroglial restricted promotor element” is a nucleotide sequence which, when operably linked with a polynucleotide which encodes or specifies a gene product, causes the gene product to be produced only substantially in a neuroglial cell. An “astrocyte specific promoter” includes a promoter which, when operably linked with a polynucleotide which encodes or specifies a gene product, causes the gene product to be produced only substantially in astrocytes.

A “temporally-restricted promoter or regulatory sequence” or “temporally restricted promotor element” is a nucleotide sequence which, when operably linked with a polynucleotide which encodes or specifies a gene product, causes the gene product to be produced in a living human cell substantially only if the cell is at a particular developmental stage or is subjected to an agent which induces the expression of the promoter, e.g., tetracycline or tamoxifen.

A “transcribed polynucleotide” is a polynucleotide (e.g. an RNA, a cDNA, or an analog of one of an RNA or cDNA) which is complementary to or homologous with all or a portion of a mature RNA made by transcription of a marker of the invention and normal post-transcriptional processing (e.g. splicing), if any, of the transcript, and reverse transcription of the transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “homology” or “identity,” as used interchangeably herein, refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantial homology,” as used herein, refers to homology of at least 50%, more preferably, 60%, 70%, 80%, 90%, 95% or more.

A marker is “fixed” to a substrate if it is covalently or non-covalently associated with the substrate such the substrate can be rinsed with a fluid (e.g. standard saline citrate, pH 7.4) without a substantial fraction of the marker dissociating from the substrate.

As used herein, a “naturally-occurring” nucleic acid molecule refers to an RNA or DNA molecule having a nucleotide sequence that occurs in nature (e.g. encodes a natural protein).

A neurological disease, disorder, or condition is “modulated”, e.g., “inhibited” if at least one symptom of the neurological disease, disorder, or condition is alleviated, terminated, slowed, or prevented. As used herein, a neurological disease, disorder, or condition is also “inhibited” if relapse, recurrence or metastasis of the neurological disease, disorder, or condition, e.g., a tumor, e.g., a glioma, is reduced, slowed, delayed, or prevented.

A kit is any manufacture (e.g. a package or container) comprising at least one reagent, e.g. a probe, for specifically detecting a marker of the invention, the manufacture being promoted, distributed, or sold as a unit for performing the methods of the present invention.

### II. USES OF THE INVENTION

The present invention is based, in part, on the identification of markers involved in neural cell survival and/or neural cell homeostasis, e.g., markers preferentially expressed in neuroglia, e.g., astrocytes, which have an altered amount, structure, and/or activity in cells afflicted with a neurological disease, disorder, or condition as compared to normal (i.e., non-afflicted or control) cells. The markers of the invention correspond to DNA, cDNA, RNA, and polypeptide molecules which can be detected in one or both of normal and afflicted cells.

The amount, structure, and/or activity, e.g., the presence, absence, copy number, expression level, protein level, protein activity, presence of mutations, e.g., mutations which affect activity of the marker (e.g., substitution, deletion, or addition mutations), and/or methylation status, of one or more of these markers in a sample, e.g., a sample containing tissue or cells, e.g., neuroglial tissue or cells, e.g., astrocytes, whole blood, serum, plasma, buccal scrape, saliva, spinal fluid, cerebrospinal fluid, urine, stool, is herein correlated with the disease state of the tissue. The invention thus provides compositions, kits, and methods for assessing the disease state of cells (e.g. cells obtained from a non-human, cultured non-human cells, and in vivo cells) as well as methods for treatment, prevention, and/or inhibition of a neurological disease, disorder or condition using a modulator, e.g., an agonist or antagonist, of a marker of the invention.

The compositions, kits, and methods of the invention have the following uses, among others:
1) assessing whether a subject is afflicted with a neurological disease, disorder or condition;
2) assessing the stage of a neurological disease, disorder or condition, e.g., a central nervous system tumor, in a human subject;
3) assessing the grade of a tumor, e.g., a glioma, in a subject;
4) assessing the benign or malignant nature of a tumor, e.g., a glioma, in a subject;
5) assessing the metastatic potential of a tumor, e.g., a glioma, in a subject;
6) assessing the histological type of a tumor, e.g., a glioma, in a subject;
7) assessing the clinical outcome of a subject afflicted with a tumor, e.g., a glioma;
8) predicting responsiveness of a subject afflicted with a tumor, e.g., a glioma, to treatment;
9) identifying the appropriate treatment of a subject afflicted with a tumor, e.g., a glioma;
10) making antibodies, antibody fragments or antibody derivatives that are useful for treating a neurological disease, disorder or condition and/or assessing whether a subject is afflicted with a neurological disease, disorder or condition;
11) assessing the presence of neuroglia cells, e.g., astrocytes, in a sample;
12) assessing the efficacy of one or more test compounds for inhibiting a neurological disease, disorder or condition in a subject;
13) assessing the efficacy of a therapy for inhibiting a neurological disease, disorder or condition in a subject;
14) monitoring the progression of a neurological disease, disorder or condition in a subject;
15) selecting a composition or therapy for inhibiting a neurological disease, disorder or condition, e.g., in a subject;
16) treating a subject afflicted with a neurological disease, disorder or condition;
17) modulating, e.g., inhibiting, a neurological disease, disorder or condition in a subject;
18) modulating neural homeostasis in a subject;
19) modulating neural cell survival in a subject;
20) assessing the carcinogenic potential of a test compound; and
21) preventing the onset of a neurological disease, disorder or condition in a subject at risk for developing a neurological disease, disorder or condition.

The invention thus includes a method of assessing whether a subject is afflicted with a neurological disease, disorder or condition or is at risk for developing a neurological disease, disorder or condition. This method comprises comparing the amount, structure, and/or activity, e.g., the presence, absence, copy number, expression level, protein level, protein activity, presence of mutations, e.g., mutations which affect activity of the marker (e.g., substitution, deletion, or addition mutations), and/or methylation status, of a marker in a subject sample with the normal level. A significant difference between the amount, structure, or activity of the marker in the subject sample and the normal level is an indication that the subject is afflicted with a neurological disease, disorder or condition.

The marker is selected from the group consisting of the markers listed in Table 2. In one embodiment, the marker is selected from the markers listed in Table 5 or Table 7. Table 2 lists the markers which are differentially expressed in samples histologically identified as neuroglia, e.g., astrocytes. Table 2 also lists the Locus ID No, MGI Accession Number, Affymetrix probe-set accession number, and GenBank accession number for the nucleic acid sequence and the amino acid sequence of each of the markers. The amino acid sequence of the each of the markers listed in Table 2 is attached herewith as Appendix B. The nucleic acid sequence of the each of the markers listed in Table 2 is attached herewith as Appendix A. Table 5 and Table 7 list a subset of markers from Table 2 that are preferred markers with respect to the methods and compositions described herein. Although one or more molecules corresponding to the markers listed in Table 2, Table 5 and Table 7 may have been described by others, the significance of these markers with regard to astrocyte-specific expression and with regard to their significance in diagnosing, prognosing, characterizing, treating, and/or preventing a neurological disease, disorder, or condition, in a subject has not previously been identified.

Any marker or combination of markers listed in Table 2 may be used in the compositions, kits, and methods of the present invention. In general, it is preferable to use markers for which the difference between the amount, e.g., level of expression or copy number, and/or activity of the marker in cells afflicted with a neurological disease, disorder or condition, and the amount, e.g., level of expression or copy number, and/or activity of the same marker in normal cells, is as great as possible. Although this difference can be as small as the limit of detection of the method for assessing amount and/or activity of the marker, it is preferred that the difference be at least greater than the standard error of the assessment method, and preferably a difference of at least 2-, 3-, 4-, 5-, 6-, 7-, 8-, 9-, 10-, 15-, 20-, 25-, 100-, 500-, 1000-fold or greater than the amount, e.g., level of expression or copy number, and/or activity of the same marker in normal tissue.

It is understood that by routine screening of additional subject samples using one or more of the markers of the invention, it will be realized that certain of the markers have altered amount, structure, and/or activity in various neurological diseases, disorders or conditions, including tumors, e.g., gliomas. It is also understood that certain markers of the invention will be associated with high grade tumors, e.g., gliomas, and certain markers of the invention will be associated with low grade tumors, e.g., gliomas.

For example, it will be confirmed that some of the markers of the invention have altered amount, structure, and/or activity in some, i.e., 10%, 20%, 30%, or 40%, or most (i.e. 50% or more) or substantially all (i.e. 80% or more) neurological diseases, disorders, or conditions. Furthermore, certain of the markers of the invention are associated with a cancer or tumor of the central nervous system, of various histologic subtypes or grades.

In addition, as a greater number of subject samples are assessed for altered amount, structure, and/or activity of the markers or altered expression of the invention and the outcomes of the individual subjects from whom the samples were obtained are correlated, it will also be confirmed that markers have altered amount, structure, and/or activity of certain of the markers or altered expression of the invention are strongly correlated with a cancer or tumor of the central nervous system, e.g., a malignant tumor, and that altered expression of other markers of the invention are strongly correlated with a neurological disease, disorder, or condition, e.g., a cancer or tumor of the central nervous system, e.g., a benign tumor or premalignant state. The compositions, kits, and methods of the invention are thus useful for characterizing one or more of the stage, grade, histological type, and benign/premalignant/malignant nature of, e.g., a cancer or tumor, in a subject.

When the compositions, kits, and methods of the invention are used for characterizing one or more of the stage, grade, histological type, and benign/premalignant/malignant nature of a central nervous system tumor, in a subject, it is preferred that the marker or panel of markers of the invention be selected such that a positive result is obtained in at least about 20%, and preferably at least about 40%, 60%, or 80%, and more preferably, in substantially all, subjects afflicted with a central nervous system tumor, of the corresponding stage, grade, histological type, or benign/premalignant/malignant nature. Preferably, the marker or panel of markers of the invention is selected such that a PPV (positive predictive value) of greater than about 10% is obtained for the general population (more preferably coupled with an assay specificity greater than 99.5%).

When a plurality of markers of the invention are used in the compositions, kits, and methods of the invention, the amount, structure, and/or activity of each marker or level of expression or copy number can be compared with the normal amount, structure, and/or activity of each of the plurality of markers or level of expression or copy number, in non-afflicted, e.g., control, samples of the same type, either in a single reaction mixture (i.e. using reagents, such as different fluorescent probes, for each marker) or in individual reaction mixtures corresponding to one or more of the markers.

In one embodiment, a significantly altered or modulated amount, structure, and/or activity of more than one of the plurality of markers, in the sample, relative to the corresponding normal levels, is an indication that the subject is afflicted with a neurological disease, disorder or condition. For example, a significantly lower copy number in the sample of each of the plurality of markers, relative to the corresponding normal levels or copy number, is an indication that the subject is afflicted with a neurological disease, disorder or condition. In yet another embodiment, a significantly enhanced copy number of one or more markers and a significantly lower level of expression or copy number of one or more markers in a sample relative to the corresponding normal levels, is an indication that the subject is afflicted with a neurological disease, disorder or condition. Also, for example, a significantly enhanced copy number in the sample of each of the plurality of markers, relative to the corresponding normal copy number, is an indication that the subject is afflicted with a neurological disease, disorder or condition. In yet another embodiment, a significantly enhanced copy number of one or more markers and a significantly lower copy number of one or more markers in a sample relative to the corresponding normal levels, is an indication that the subject is afflicted with a neurological disease, disorder or condition.

When a plurality of markers are used, it is preferred that 2, 3, 4, 5, 8, 10, 12, 15, 20, 30, or 50 or more individual markers be used or identified, wherein fewer markers are preferred.

It is recognized that the compositions, kits, and methods of the invention will be of particular utility to subjects having an enhanced risk of developing a neurological disease, disorder or condition, and their medical advisors. Subjects recognized as having an enhanced risk of developing a neurological disease, disorder or condition, include, for example, subjects having a familial history of a neurological disease, disorder or condition, subjects identified as having a mutant oncogene (i.e. at least one allele), and subjects of advancing age.

A modulation, e.g., an alteration, e.g. copy number, amount, structure, and/or activity of a marker in normal (i.e. non-afflicted) human tissue can be assessed in a variety of ways. In one embodiment, the normal level of expression or copy number is assessed by assessing the level of expression and/or copy number of the marker in a portion of cells which appear to be non-afflicted and by comparing this normal level of expression or copy number with the level of expression or copy number in a portion of the cells which are suspected of being diseased or afflicted. For example, when a medical procedure reveals the presence of a tumor in one region of the CNS, the normal level of expression or copy number of a marker may be assessed using the non-affected portion of the CNS, and this normal level of expression or copy number may be compared with the level of expression or copy number of the same marker in an affected portion (i.e., the tumor) of the CNS. Alternately, and particularly as further information becomes available as a result of routine performance of the methods described herein, population-average values for “normal” copy number, amount, structure, and/or activity of the markers of the invention may be used. In other embodiments, the “normal” copy number, amount, structure, and/or activity of a marker may be determined by assessing copy number, amount, structure, and/or activity of the marker in a subject sample obtained from a non-neurological disease-, disorder- or condition-afflicted subject, from a subject sample obtained from a subject before the suspected onset of a neurological disease, disorder, or condition in the subject, from archived subject samples, and the like.

The invention includes compositions, kits, and methods for assessing the presence of neuroglial cells, e.g., astrocytes, in a sample (e.g. an archived tissue sample or a sample obtained from a subject). These compositions, kits, and methods are substantially the same as those described above, except that, where necessary, the compositions, kits, and methods are adapted for use with certain types of samples. For example, when the sample is a parafinized, archived human tissue sample, it may be necessary to adjust the ratio of compounds in the compositions of the invention, in the kits of the invention, or the methods used. Such methods are well known in the art and within the skill of the ordinary artisan.

The invention thus includes a kit for assessing the presence of neuroglial cells, e.g., astrocytes, (e.g. in a sample such as a subject sample) as well as a kit for assessing the amount or activity of a marker of the invention in a sample. The kit may comprise one or more reagents capable of identifying a marker of the invention, e.g., binding specifically with a nucleic acid or polypeptide corresponding to a marker of the invention. Suitable reagents for binding with a polypeptide corresponding to a marker of the invention include antibodies, antibody derivatives, antibody fragments, and the like. Suitable reagents for binding with a nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kits of the invention may optionally comprise additional components useful for performing the methods of the invention. By way of example, the kit may comprise fluids (e.g., SSC buffer) suitable for annealing complementary nucleic acids or for binding an antibody with a protein with which it specifically binds, one or more sample compartments, an instructional material which describes performance of a method of the invention, a sample of normal cells, a sample of neuroglial cells, and the like.

A kit of the invention may comprise a reagent useful for determining protein level or protein activity of a marker. In another embodiment, a kit of the invention may comprise a reagent for determining methylation status of a marker, or may comprise a reagent for determining alteration of structure of a marker, e.g., the presence of a mutation.

The invention also includes a method of making an isolated hybridoma which produces an antibody useful in methods and kits of the present invention. A protein corresponding to a marker of the invention may be isolated (e.g., by purification from a cell in which it is expressed or by transcription and translation of a nucleic acid encoding the protein in vivo or in vitro using known methods) and a vertebrate, preferably a mammal such as a mouse, rat, rabbit, or sheep, is immunized using the isolated protein. The vertebrate may optionally (and preferably) be immunized at least one additional time with the isolated protein, so that the vertebrate exhibits a robust immune response to the protein. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein. The invention also includes hybridomas made by this method and antibodies made using such hybridomas.

The invention also includes a method of assessing the efficacy of a test compound for modulating, e.g., inhibiting, a neurological disease, disorder, or condition. As described above, differences in the amount, structure, and/or activity of the markers of the invention, or level of expression of the invention, or copy number, correlate with the afflicted state of cells. Although it is recognized that changes in the levels of amount, e.g., expression or copy number, structure, and/or activity of certain of the markers or expression or copy number of the invention likely result from the afflicted state of cells, it is likewise recognized that changes in the amount may induce, maintain, and promote the afflicted state. Thus, compounds which modulate, e.g., inhibit, a neurological disease, disorder, or condition, in a subject may cause a change, e.g., a change in expression and/or activity of one or more of the markers of the invention to a level nearer the normal level for that marker (e.g., the amount, e.g., expression, and/or activity for the marker in non-afflicted cells).

This method thus comprises comparing amount, e.g., expression, and/or activity of a marker in a first cell sample and maintained in the presence of the test compound and amount, e.g., expression, and/or activity of the marker in a second cell sample and maintained in the absence of the test compound. A significant modulation in the amount, e.g., expression, and/or activity of a marker, or a significant decrease in the amount, e.g., expression, and/or activity of a marker listed in Table 2, is an indication that the test compound modulates a neurological disease, disorder, or condition. The cell samples may, for example, be aliquots of a single sample of normal cells obtained from a subject, pooled samples of normal cells obtained from a subject, cells of a normal cell lines, aliquots of a single sample of afflicted cells obtained from a subject, pooled samples of afflicted cells obtained from a subject, cells of a neuroglial cell line, cells from an animal model of a neurological disease, disorder, or condition, or the like. In one embodiment, the samples are neuroglial cells obtained from a subject and a plurality of compounds known to be effective for modulating various neurological diseases, disorders, or conditions, are tested in order to identify the compound which is likely to best modulate the a neurological disease, disorder, or condition in the subject.

This method may likewise be used to assess the efficacy of a therapy, e.g., chemotherapy, radiation therapy, surgery, or any other therapeutic approach useful for modulating a neurological disease, disorder, or condition in a subject. In this method, the amount, e.g., expression, and/or activity of one or more markers of the invention in a pair of samples (one subjected to the therapy, the other not subjected to the therapy) is assessed. As with the method of assessing the efficacy of test compounds, if the therapy induces a significant modulation in the amount, e.g., expression, and/or activity of a marker listed in Table 2 then the therapy is efficacious for modulating a neurological disease, disorder, or condition. As above, if samples from a selected subject are used in this method, then alternative therapies can be assessed in vitro in order to select a therapy most likely to be efficacious for modulating a neurological disease, disorder, or condition in the subject.

This method may likewise be used to monitor the progression of a neurological disease, disorder, or condition in a subject, wherein if a sample in a subject has a significant modulation in the amount, e.g., expression, and/or activity of a marker listed in Table 2 during the progression of a neurological disease, disorder, or condition, e.g., at a first point in time and a subsequent point in time, then the neurological disease, disorder, or condition has been modulated, e.g., improved. In yet another embodiment, between the first point in time and a subsequent point in time, the subject has undergone treatment, e.g., chemotherapy, radiation therapy, surgery, or any other therapeutic approach useful for inhibiting a neurological disease, disorder, or condition, has completed treatment, or is in remission.

As described herein, a neurological disease, disorder, or condition in a subject is associated with a modulation in neural cell survival and/or neural homeostasis which is associated with modulation in the amount, e.g., expression, and/or activity of one or more markers listed in Table 2. While, as discussed above, some of these changes in amount, e.g., expression, and/or activity number result from occurrence of the neurological disease, disorder, or condition, others of these changes induce, maintain, and promote the disease state of afflicted cells. Thus, a neurological disease, disorder, or condition characterized by a modulation, e.g., an increase, in the amount, e.g., expression, and/or activity of one or more markers listed in Table 2 (e.g., a marker that was shown to be increased in a neurological disease, disorder, or condition), can be modulated, e.g., inhibited, by modulating, e.g., inhibiting, the amount, e.g., expression, and/or activity of those markers. Likewise, a neurological disease, disorder, or condition characterized by a modulation, e.g., a decrease, in the amount, e.g., expression, and/or activity of one or more markers listed in Table 2 (e.g., a marker that was shown to be decreased in neurological disease, disorder, or condition), can be modulated, e.g., enhanced, by modulating, e.g., enhancing, amount, e.g., expression, and/or activity of those markers.

Amount and/or activity of a marker listed in Table 2 (e.g., a marker that was shown to be modulated, e.g., increased, in a neurological disease, disorder, or condition), can be modulated e.g., decreased, in a number of ways generally known in the art. For example, an antisense oligonucleotide can be provided to the afflicted cells in order to inhibit transcription, translation, or both, of the marker(s). An RNA interfering agent, e.g., an siRNA molecule, which is targeted to a marker listed in Table 2, can be provided to the afflicted cells in order to inhibit expression of the target marker, e.g., through degradation or specific post-transcriptional gene silencing (PTGS) of the messenger RNA (mRNA) of the target marker. Alternately, a polynucleotide encoding an antibody, an antibody derivative, or an antibody fragment, e.g., a fragment capable of binding an antigen, and operably linked with an appropriate promoter or regulator region, can be provided to the cell in order to generate intracellular antibodies which will inhibit the function, amount, and/or activity of the protein corresponding to the marker(s). Conjugated antibodies or fragments thereof, e.g., chemolabeled antibodies, radiolabeled antibodies, or immunotoxins targeting a marker of the invention may also be administered to treat, prevent or inhibit a neurological disease, disorder, or condition.

A small molecule may also be used to modulate expression and/or activity of a marker listed in Table 2. In one embodiment, a small molecule functions to disrupt a protein-protein interaction between a marker of the invention and a target molecule or ligand, thereby modulating, e.g., increasing or decreasing, the activity of the marker.

Using the methods described herein, a variety of molecules, particularly including molecules sufficiently small that they are able to cross the cell membrane, can be screened in order to identify molecules which modulate, e.g., inhibit, the amount and/or activity of the marker(s). The compound so identified can be provided to the subject in order to modulate, e.g., inhibit, the amount and/or activity of the marker(s) in the afflicted cells of the subject.

Amount and/or activity of a marker listed in Table 2 (e.g., a marker that was shown to be decreased in a neurological disease, disorder, or condition) can be modulated, e.g., enhanced, in a number of ways generally known in the art. For example, a polynucleotide encoding the marker and operably linked with an appropriate promoter/regulator region can be provided to cells of the subject in order to induce enhanced expression and/or activity of the protein (and mRNA) corresponding to the marker therein. Alternatively, if the protein is capable of crossing the cell membrane, inserting itself in the cell membrane, or is normally a secreted protein, then amount and/or activity of the protein can be enhanced by providing the protein (e.g. directly or by way of the bloodstream) to afflicted cells in the subject. A small molecule may also be used to modulate, e.g., increase, expression or activity of a marker listed in Table 2 (e.g., a marker that was shown to be decreased in a neurological disease, disorder, or condition). Furthermore, in another embodiment, a modulator of a marker of the invention, e.g., a small molecule, may be used, for example, to re-express a silenced gene, e.g., a tumor suppressor, in order to treat or prevent a neurological disease, disorder, or condition, e.g., a central nervous system tumor. For example, such a modulator may interfere with a DNA binding element or a methyltransferase.

As described above, neural cell survival and neural cell homeostasis and the afflicted state of human cells is correlated with changes in the amount and/or activity of the markers of the invention. Thus, compounds which induce increased expression or activity of one or more of the markers listed in Table 2 (e.g., a marker that was shown to be increased in a neurological disease, disorder, or condition), decreased amount and/or activity of one or more of the markers listed in Table 2 (e.g., a marker that was shown to be decreased in neurological disease, disorder, or condition), can induce cell carcinogenesis or a neurological disease, disorder or condition. The invention also includes a method for assessing the human cell carcinogenic potential of a test compound. This method comprises maintaining separate aliquots of human cells in the presence and absence of the test compound. Expression or activity of a marker of the invention in each of the aliquots is compared. A significant modulation, e.g., a significant increase, in the amount and/or activity of a marker listed in Table 2 (e.g., a marker that was shown to be increased in a neurological disease, disorder, or condition), or a significant modulation, e.g., a significant decrease in the amount and/or activity of a marker listed in Table 2 (e.g., a marker that was shown to be decreased in a neurological disease, disorder, or condition), in the aliquot maintained in the presence of the test compound (relative to the aliquot maintained in the absence of the test compound) is an indication that the test compound possesses human cell carcinogenic potential or the ability to induce a neurological disease, disorder or condition. The relative disease causing potential of various test compounds can be assessed by comparing the degree of enhancement or inhibition of the amount and/or activity of the relevant markers, by comparing the number of markers for which the amount and/or activity is modulated, e.g., enhanced or inhibited, or by comparing both.

### III. ISOLATED NUCLEIC ACID MOLECULES

One aspect of the invention pertains to nucleic acid molecules that correspond to a marker of the invention, including nucleic acids which encode a polypeptide corresponding to a marker of the invention or a portion of such a polypeptide. Nucleic acid molecules of the invention also include nucleic acid molecules sufficient for use as hybridization probes to identify nucleic acid molecules that correspond to a marker of the invention, including nucleic acid molecules which encode a polypeptide corresponding to a marker of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

In one embodiment, a nucleic acid molecule of the invention is an isolated nucleic acid molecule. An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A nucleic acid molecule of the present invention, e.g., a nucleic acid molecule encoding a protein corresponding to a marker listed in Table 2, can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, a nucleic acid molecule of the invention comprises a nucleic acid molecule which has a nucleotide sequence complementary to the nucleotide sequence of a nucleic acid corresponding to a marker of the invention or to the nucleotide sequence of a nucleic acid encoding a protein which corresponds to a marker of the invention. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker of the invention or which encodes a polypeptide corresponding to a marker of the invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acid molecules encoding a protein which corresponds to a marker of the invention, and thus encode the same protein.

In addition to the nucleotide sequences described in Table 2, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, a nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a nucleic acid molecule corresponding to a marker of the invention or to a nucleic acid molecule encoding a protein corresponding to a marker of the invention. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins which correspond to the markers of the invention, yet retain biological activity. In one embodiment, such a protein has an amino acid sequence that is at least about 40% identical, 50%, 60%, 70%, 80%, 90%, 95%, or 98% identical to the amino acid sequence of one of the proteins which correspond to the markers of the invention.

A nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded cDNA molecule corresponding to a marker of the invention or complementary to an mRNA sequence corresponding to a marker of the invention. Accordingly, an antisense nucleic acid molecule of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide corresponding to a selected marker of the invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention include direct injection at a tissue site or infusion of the antisense nucleic acid into an appropriately-associated body fluid, e.g., cerebrospinal fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acid molecules having at least one region which is complementary to a nucleic acid molecule of the invention, such that the molecular beacon is useful for quantitating the presence of the nucleic acid molecule of the invention in a sample. A “molecular beacon” nucleic acid is a nucleic acid molecule comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid molecules are not annealed with one another, fluorescence of the fluorophore is quenched to a lesser degree. Molecular beacon nucleic acid molecules are described, for example, in U.S. Pat. No. 5,876,930.

In another embodiment, a nucleic acid molecule contains sequences which naturally flank the nucleic acid (i.e., sequences located at the 5′ and 3′ ends of the nucleic acid) in the genomic DNA of the organism from which the nucleic acid is derived. In various embodiments, the isolated nucleic acid molecule can contain about 100 kB, 50 kB, 25 kB, 15 kB, 10 kB, 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. For example, in various embodiments, the nucleic acid molecules of the invention contain temporal and spatial regulatory elements (e.g., elements that restrict the expression of the markers of the invention to neuroglia, e.g., astrocytes, or restrict the expression of the marker of the invention to a specific developmental stage), that are proximal or 5′ to the initiation signal, e.g., the initiating ATG codon. Moreover, a nucleic acid molecule can be substantially free of other cellular material or culture medium when produced by recombinant techniques, or substantially free of chemical precursors or other chemicals when chemically synthesized.

Nucleic acid molecules of the invention corresponding to temporal and spatial regulatory elements, e.g., temporal and spatial promotors, of a marker of the invention can be used to construct recombinant expression vectors. The identification of temporal and spatial regulatory elements (e.g., neuroglial specific regulatory elements such as astrocyte specific regulatory elements), can be performed by creating recombinant expression vectors containing nucleic acid molecules with putative temporal and spatial regulatory elements operably linked to sites of inducible recombination, such as, for example, lox sites, e.g., loxP sites, and optionally further operably linked to a reporter sequence, such as, for example, LacZ, GFP, and EGFP. Such recombinant expression vectors can be used to generate transgenic animals, the cells of which can subsequently be examined for temporal and spatial restriction of the reporter sequence, e.g., expression substantially only in neuroglial cells, e.g., astrocytes, to identify nucleic acid molecules of the invention corresponding to temporal and spatial regulatory elements.

Such transgenic animals as described above (and in Section V) are not only useful for identifying spatial and temporal regulatory elements, but are also useful for studying the function and/or activity of the polypeptide corresponding to the marker of the invention, for identifying and/or evaluating modulators of polypeptide activity, as well as in pre-clinical testing of therapeutics or diagnostic agents, for marker discovery or evaluation, e.g., therapeutic and diagnostic marker discovery or evaluation, or as surrogates of drug efficacy and specificity. Furthermore, such animals are useful for the investigation of the effect, e.g., physiological effect, of a temporal and spatial restriction of a gene of interest. For example, a transgene may cause lethality due to the requirement of the gene at a particular point in development. However, the same transgene under the control of a spatially and/or temporally regulated promoter may be induced subsequent to the point in time that loss of the gene causes lethality and/or in a specific tissue that does not cause lethality. Alternatively, a gene that is ubiquitously expressed in normal cells, e.g., cells not afflicted with a disease, disorder, or condition, may be preferentially overexpressed or misexpressed in a disease, disorder, or condition, such as, for example, a neurological disease, disorder, or condition, such as a cancer of the central nervous system. For example, epidermal growth factor receptor (EGFR) is expressed in many tissues of the embryo and adult, but has been shown to be overexpressed specifically in neuroglial cells, e.g., astrocytes, in a neurological disease, disorder and condition. Operably linking EGFR to a spatially restricted promoter of the invention, e.g., an astrocyte-specific promoter, and further operably linking an inducible promoter, such as, for example, the CRE:estrogen receptor, will allow controlled expression, e.g., inducible expression, of EGFR in specific cell types, e.g., neuroglia, e.g., astrocytes, in order to more closely model a neurological disease, disorder, or condition for the study of the progression, maintenance, and/or response to treatment of a neurological disease, disorder, or condition.

### IV. ISOLATED PROTEINS AND ANTIBODIES

One aspect of the invention pertains to isolated proteins which correspond to individual markers of the invention, and biologically active portions thereof, as well as polypeptide fragments suitable for use as immunogens to raise antibodies directed against a polypeptide corresponding to a marker of the invention. In one embodiment, the native polypeptide corresponding to a marker can be isolated from cells or tissue sources by an appropriate purification scheme using standard protein purification techniques. In another embodiment, polypeptides corresponding to a marker of the invention are produced by recombinant DNA techniques. Alternative to recombinant expression, a polypeptide corresponding to a marker of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide corresponding to a marker of the invention include polypeptides comprising amino acid sequences sufficiently identical to or derived from the amino acid sequence of the protein corresponding to the marker (e.g., the protein encoded by the nucleic acid molecules listed in Table 2), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have an amino acid sequence of a protein encoded by a nucleic acid molecule listed in Table 2. Other useful proteins are substantially identical (e.g., at least about 40%, preferably 50%, 60%, 70%, 80%, 90%, 95%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins corresponding to a marker of the invention. As used herein, a “chimeric protein” or “fusion protein” comprises all or part (preferably a biologically active part) of a polypeptide corresponding to a marker of the invention operably linked to a heterologous polypeptide (i.e., a polypeptide other than the polypeptide corresponding to the marker). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide corresponding to a marker of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide corresponding to a marker of the invention can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide corresponding to a marker of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the polypeptides corresponding to individual markers of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

An isolated polypeptide corresponding to a marker of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the polypeptides of the invention, and encompasses an epitope of the protein such that an antibody raised against the peptide forms a specific immune complex with a marker of the invention to which the protein corresponds. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions.

An immunogen typically is used to prepare antibodies by immunizing a suitable (i.e. immunocompetent) subject such as a rabbit, goat, mouse, or other mammal or vertebrate.

An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al, pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al (1988) J. Immunol. 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

An antibody, antibody derivative, or fragment thereof, which specifically binds a marker of the invention which is modulated in a neurological disease, disorder, or condition (e.g., a marker set forth in Table 2), may be used to inhibit activity of a marker, e.g., a marker set forth in Table 2, and therefore may be administered to a subject to treat, inhibit, or prevent cancer in the subject. Furthermore, conjugated antibodies may also be used to treat, inhibit, or prevent cancer in a subject. Conjugated antibodies, preferably monoclonal antibodies, or fragments thereof, are antibodies which are joined to drugs, toxins, or radioactive atoms, and used as delivery vehicles to deliver those substances directly to cancer cells. The antibody, e.g., an antibody which specifically binds a marker of the invention (e.g., a marker listed in Table 2), is administered to a subject and binds the marker, thereby delivering the toxic substance to the afflicted cell, minimizing damage to normal cells in other parts of the body.

Conjugated antibodies are also referred to as “tagged,” “labeled,” or “loaded.” Antibodies with chemotherapeutic agents attached are generally referred to as chemolabeled. Antibodies with radioactive particles attached are referred to as radiolabeled, and this type of therapy is known as radioimmunotherapy (RIT). Aside from being used to treat cancer, radiolabeled antibodies can also be used to detect areas of cancer spread in the body. Antibodies attached to toxins are called immunotoxins.

Immunotoxins are made by attaching toxins (e.g., poisonous substances from plants or bacteria) to monoclonal antibodies. Immunotoxins may be produced by attaching monoclonal antibodies to bacterial toxins such as diphtherial toxin (DT) or pseudomonal exotoxin (PE40), or to plant toxins such as ricin A or saporin.

An antibody directed against a polypeptide corresponding to a marker of the invention (e.g., a monoclonal antibody) can be used to isolate the polypeptide by standard techniques, such as affinity chromatography or immunoprecipitation. Moreover, such an antibody can be used to detect the marker (e.g., in a cellular lysate or cell supernatant) in order to evaluate the level and pattern of expression of the marker. The antibodies can also be used diagnostically to monitor protein levels in tissues or body fluids (e.g. in an ovary-associated body fluid) as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### V. RECOMBINANT EXPRESSION VECTORS AND HOST CELLS

Another aspect of the invention pertains to vectors, preferably expression vectors, containing a nucleic acid encoding a polypeptide corresponding to a marker of the invention (or a portion of such a polypeptide). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide corresponding to a marker of the invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol. 185, Academic Press, San Diego, Calif., 1991). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf9 cells) include the pAc series (Smith et al., 1983, Mol. Cell. Biol. 3:2156-2165) and the pVL series (Lucklow and Summers, 1989, Virology 170:31-39).

In yet another embodiment, a nucleic acid of the invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, e.d. Nouer, L., CRC, Boca Raton, Fla., pp 167-220), hormones (see e.g., Lee et al. (1981) Nature 294:228-232; Hynes et al. (1981) Proc. Natl. Acad. Sci. USA 78:2038-2042; Klock et al. (1987) Nature 329:734-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313). Accordingly, in another embodiment, the invention provides a recombinant expression vector in which DNA corresponding to a marker of the invention is operatively linked to an inducible eukaryotic promoter, thereby allowing for inducible expression of a protein corresponding to a marker of the invention in eukaryotic cells.

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., 1986, Trends in Genetics, Vol. 1(1).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide corresponding to a marker of the invention. Accordingly, the invention further provides methods for producing a polypeptide corresponding to a marker of the invention using the host cells of the invention. In one embodiment, the method comprises culturing the host cell of invention (into which a recombinant expression vector encoding a polypeptide of the invention has been introduced) in a suitable medium such that the marker is produced. In another embodiment, the method further comprises isolating the marker polypeptide from the medium or the host cell.

The host cells of the invention can also be used to produce nonhuman transgenic animals. For example, in one embodiment, a host cell of the invention is a fertilized oocyte or an embryonic stem cell into which sequences encoding a polypeptide corresponding to a marker of the invention have been introduced.

In another embodiment, a host cell of the invention is a fertilized oocyte or an embryonic stem cell into which sequences corresponding to spatially or temporally restricted promotor elements of a marker of the invention, e.g., neuroglial-specific regulatory elements, e.g., astrocyte-specific regulatory elements, operably linked to a conditional allele. As used herein, a “conditional allele” refers to a form of a transgene whose expression is regulated and/or a transgene that may be inducibly altered in function and/or structure by application, administration or expression of an exogenous reagent (e.g., Cre recombinase expression, tamoxifen treatment) or a state change (e.g., temperature change), such that the activity or abundance of the transgene, expressed transcript, or encoded gene product is changed. The Cre-lox recombination system is described in, for example, Baubonis, W. and Sauer, B. (1993) Nucl. Acids Res. 21:2025-2029; and Fukushige, S, and Sauer, B. (1992) Proc. Natl. Acad. Sci. USA 89:7905-7909) and the FLP recombinase-FRT target system (e.g., as described in Dang, D. T. and Perrimon, N. (1992) Dev. Genet. 13:367-375; and Fiering, S. et al. (1993) Proc. Natl. Acad. Sci. USA 90:8469-8473). Additionally, conditional alleles can be generated utilizing tetracycline-regulated inducible homologous recombination systems, such as described in PCT Publication No. WO 94/29442 and PCT Publication No. WO 96/01313 or the FLP recombinase system of Saccharomyces cerevisiae (O'Gorman et al., 1991, Science 251:1351-1355).

In certain embodiments of the invention, the spatially or temporally restricted promotor elements e.g., neuroglial specific promotor elements, e.g., astrocyte specific promotor elements, operably linked to Cre, are further operably linked to an inducible fusion protein, such as, for example, the estrogen receptor (ERT2), whose protein product is a fusion of Cre recombinase and a mutant mouse estrogen receptor ligand binding domain that cannot bind estrogen at physiologic concentrations, but does bind tamoxifen. The ubiquitously-expressed fusion protein is restricted to the cytoplasm in the absence of tamoxifen; upon binding to tamoxifen, it becomes translocated to the nucleus as described in, for example, Leone D P, et al. (2003) Mol Cell Neurosci. 22:430-40.

In yet another embodiment of the invention, the spatially or temporally restricted promotor elements are operably linked to sites of inducible recombination, e.g., lox sites, e.g., loxP sites, and optionally further operably linked to a reporter sequence, e.g., lacZ, GFP, EGFP, as described above.

Such host cells can then be used to create non-human transgenic animals in which exogenous sequences encoding a marker protein or spatially or temporally restricted promotor elements of the invention have been introduced into their genome or homologous recombinant animals in which endogenous gene(s) encoding a polypeptide corresponding to a marker of the invention sequences have been altered.

As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal. Transgenic animals also include inducible transgenic animals, such as those described in, for example, Chan I. T., et al. (2004) J Clin Invest. 113(4):528-38 and Chin L. et al (1999) Nature 400(6743):468-72.

A transgenic animal of the invention can be created by introducing a nucleic acid encoding a polypeptide corresponding to a marker of the invention into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, Cold Spring Harbor Laboratory Press, Cold Spring Harbor, N.Y., 1986. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes, and will be appreciated by the skilled artisan to be required in order to generate transgenic animals carrying conditional alleles.

Clones of the non-human transgenic animals described herein can also be produced according to the methods described in Wilmut et al. (1997) Nature 385:810-813 and PCT Publication NOS. WO 97/07668 and WO 97/07669.

### VI. METHODS OF TREATMENT

The present invention provides for both prophylactic and therapeutic methods of treating a subject, e.g., a human, who has or is at risk of (or susceptible to) a neurological disease, disorder, or condition. As used herein, “treatment” of a subject includes the application or administration of a therapeutic agent to a subject, or application or administration of a therapeutic agent to a cell or tissue from a subject, who has a disease or disorder, has a symptom of a disease or disorder, or is at risk of (or susceptible to) a disease or disorder, with the purpose of curing, inhibiting, healing, alleviating, relieving, altering, remedying, ameliorating, improving, or affecting the disease or disorder, the symptom of the disease or disorder, or the risk of (or susceptibility to) the disease or disorder. As used herein, a “therapeutic agent” or “compound” includes, but is not limited to, small molecules, peptides, peptidomimetics, polypeptides, RNA interfering agents, e.g., siRNA molecules, antibodies, ribozymes, and antisense oligonucleotides.

As described herein, a neurological disease, disorder, or condition in a subject is associated with a change, e.g., an increase and/or a decrease in the amount and/or activity, or a change in the structure of one or more markers listed in Table 2. While, as discussed above, some of these changes in amount, structure, and/or activity, result from occurrence of the a neurological disease, disorder, or condition, others of these changes induce, maintain, and promote the diseased state of cells. Thus, a neurological disease, disorder, or condition, characterized by an increase in the amount and/or activity, or a change in the structure, of one or more markers listed in Table 2 (e.g., a marker that is shown to be increased in a neurological disease, disorder, or condition), can be inhibited by inhibiting amount, e.g., expression or protein level, and/or activity of those markers. Likewise, a neurological disease, disorder, or condition characterized by a decrease in the amount and/or activity, or a change in the structure, of one or more markers listed in Table 2 (e.g., a marker that is shown to be decreased in a neurological disease, disorder, or condition), can be inhibited by enhancing amount, e.g., expression or protein level, and/or activity of those markers.

Accordingly, another aspect of the invention pertains to methods for treating a subject suffering from a neurological disease, disorder, or condition. These methods involve administering to a subject a compound which modulates the amount and/or activity of one or more markers of the invention. For example, methods of treatment or prevention of a neurological disease, disorder, or condition include administering to a subject a compound which decreases the amount and/or activity of one or more markers listed in Table 2 (e.g., a marker that was shown to be increased in a neurological disease, disorder, or condition). Compounds, e.g., antagonists, which may be used to inhibit amount and/or activity of a marker listed in Table 2, to thereby treat or prevent a neurological disease, disorder, or condition include antibodies (e.g., conjugated antibodies), small molecules, RNA interfering agents, e.g., siRNA molecules, ribozymes, and antisense oligonucleotides. In one embodiment, an antibody used for treatment is conjugated to a toxin, a chemotherapeutic agent, or radioactive particles.

Methods of treatment or prevention of a neurological disease, disorder, or condition also include administering to a subject a compound which increases the amount and/or activity of one or more markers listed in Table 2 (e.g., a marker that was shown to be decreased in a neurological disease, disorder, or condition). Compounds, e.g., agonists, which may be used to increase expression or activity of a marker listed in Table 2, to thereby treat or prevent a neurological disease, disorder, or condition include small molecules, peptides, peptoids, peptidomimetics, and polypeptides.

Small molecules used in the methods of the invention include those which inhibit a protein-protein interaction and thereby either increase or decrease marker amount and/or activity. Furthermore, modulators, e.g., small molecules, which cause re-expression of silenced genes, e.g., tumor suppressors, are also included herein. For example, such molecules include compounds which interfere with DNA binding or methyltransferase activity.

An aptamer may also be used to modulate, e.g., increase or inhibit expression or activity of a marker of the invention to thereby treat, prevent or inhibit a neurological disease, disorder, or condition. Aptamers are DNA or RNA molecules that have been selected from random pools based on their ability to bind other molecules. Aptamers may be selected which bind nucleic acids or proteins.

### VII. SCREENING ASSAYS

The invention also provides methods (also referred to herein as “screening assays”) for identifying modulators, i.e., candidate or test compounds or agents (e.g., proteins, peptides, peptidomimetics, peptoids, small molecules or other drugs) which (a) bind to the marker, or (b) have a modulatory (e.g., stimulatory or inhibitory) effect on the activity of the marker or, more specifically, (c) have a modulatory effect on the interactions of the marker with one or more of its natural substrates (e.g., peptide, protein, hormone, co-factor, or nucleic acid), or (d) have a modulatory effect on the expression of the marker. Such assays typically comprise a reaction between the marker and one or more assay components. The other components may be either the test compound itself, or a combination of test compound and a natural binding partner of the marker. Compounds identified via assays such as those described herein may be useful, for example, for modulating, e.g., inhibiting, ameliorating, treating, or preventing a neurological disease, disorder, or condition.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

In one embodiment, the invention provides assays for screening candidate or test compounds which are substrates of a marker or biologically active portion thereof. In another embodiment, the invention provides assays for screening candidate or test compounds which bind to a marker or biologically active portion thereof. Determining the ability of the test compound to directly bind to a marker can be accomplished, for example, by coupling the compound with a radioisotope or enzymatic label such that binding of the compound to the marker can be determined by detecting the labeled marker compound in a complex. For example, compounds (e.g., marker substrates) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or test compounds which modulate the activity of a marker or a biologically active portion thereof. In all likelihood, the marker can, in vivo, interact with one or more molecules, such as, but not limited to, peptides, proteins, hormones, cofactors and nucleic acids. For the purposes of this discussion, such cellular and extracellular molecules are referred to herein as “binding partners” or marker “substrate”.

One necessary embodiment of the invention in order to facilitate such screening is the use of the marker to identify its natural in vivo binding partners. There are many ways to accomplish this which are known to one skilled in the art. One example is the use of the marker protein as “bait protein” in a two-hybrid assay or three-hybrid assay (see, e.g., U.S. Pat. No. 5,283,317; Zervos et al, 1993, Cell 72:223-232; Madura et al, 1993, J. Biol. Chem. 268:12046-12054; Bartel et al, 1993, Biotechniques 14:920-924; Iwabuchi et al, 1993 Oncogene 8:1693-1696; Brent WO94/10300) in order to identify other proteins which bind to or interact with the marker (binding partners) and, therefore, are possibly involved in the natural function of the marker. Such marker binding partners are also likely to be involved in the propagation of signals by the marker or downstream elements of a marker-mediated signaling pathway. Alternatively, such marker binding partners may also be found to be inhibitors of the marker.

The two-hybrid system is based on the modular nature of most transcription factors, which consist of separable DNA-binding and activation domains. Briefly, the assay utilizes two different DNA constructs. In one construct, the gene that encodes a marker protein fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a marker-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be readily detected and cell colonies containing the functional transcription factor can be isolated and used to obtain the cloned gene which encodes the protein which interacts with the marker protein.

In a further embodiment, assays may be devised through the use of the invention for the purpose of identifying compounds which modulate (e.g., affect either positively or negatively) interactions between a marker and its substrates and/or binding partners. Such compounds can include, but are not limited to, molecules such as antibodies, peptides, hormones, oligonucleotides, nucleic acids, and analogs thereof. Such compounds may also be obtained from any available source, including systematic libraries of natural and/or synthetic compounds. The preferred assay components for use in this embodiment is a cancer, marker identified herein, the known binding partner and/or substrate of same, and the test compound. Test compounds can be supplied from any source.

The basic principle of the assay systems used to identify compounds that interfere with the interaction between the marker and its binding partner involves preparing a reaction mixture containing the marker and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the marker and its binding partner. Control reaction mixtures are incubated without the test compound or with a placebo. The formation of any complexes between the marker and its binding partner is then detected. The formation of a complex in the control reaction, but less or no such formation in the reaction mixture containing the test compound, indicates that the compound interferes with the interaction of the marker and its binding partner. Conversely, the formation of more complex in the presence of compound than in the control reaction indicates that the compound may enhance interaction of the marker and its binding partner. The assay for compounds that interfere with the interaction of the marker with its binding partner may be conducted in a heterogeneous or homogeneous format. Heterogeneous assays involve anchoring either the marker or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the markers and the binding partners (e.g., by competition) can be identified by conducting the reaction in the presence of the test substance, i.e., by adding the test substance to the reaction mixture prior to or simultaneously with the marker and its interactive binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the marker or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the marker or its binding partner and drying. Alternatively, an immobilized antibody specific for the assay component to be anchored can be used for this purpose. Such surfaces can often be prepared in advance and stored.

In related embodiments, a fusion protein can be provided which adds a domain that allows one or both of the assay components to be anchored to a matrix. For example, glutathione-S-transferase/marker fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed marker or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of marker binding or activity determined using standard techniques.

Other techniques for immobilizing proteins on matrices can also be used in the screening assays of the invention. For example, either a marker or a marker binding partner can be immobilized utilizing conjugation of biotin and streptavidin. Biotinylated marker protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the corresponding partner of the immobilized assay component is exposed to the coated surface with or without the test compound. After the reaction is complete, unreacted assay components are removed (e.g., by washing) and any complexes formed will remain immobilized on the solid surface. The detection of complexes anchored on the solid surface can be accomplished in a number of ways. Where the non-immobilized component is pre-labeled, the detection of label immobilized on the surface indicates that complexes were formed. Where the non-immobilized component is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds which modulate (inhibit or enhance) complex formation or which disrupt preformed complexes can be detected.

In an alternate embodiment of the invention, a homogeneous assay may be used. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August; 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J Mol. Recognit. 11:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the marker and its binding partner.

Also within the scope of the present invention are methods for direct detection of interactions between the marker and its natural binding partner and/or a test compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et al, U.S. Pat. No. 4,868,103). Generally, this technique involves the addition of a fluorophore label on a first ‘donor’ molecule (e.g., marker or test compound) such that its emitted fluorescent energy will be absorbed by a fluorescent label on a second, ‘acceptor’ molecule (e.g., marker or test compound), which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). A test substance which either enhances or hinders participation of one of the species in the preformed complex will result in the generation of a signal variant to that of background. In this way, test substances that modulate interactions between a marker and its binding partner can be identified in controlled assays.

In another embodiment, modulators of marker expression are identified in a method wherein a cell is contacted with a candidate compound and the expression of mRNA or protein, corresponding to a marker in the cell, is determined. The level of expression of mRNA or protein in the presence of the candidate compound is compared to the level of expression of mRNA or protein in the absence of the candidate compound. The candidate compound can then be identified as a modulator of marker expression based on this comparison. For example, when expression of marker mRNA or protein is greater (statistically significantly greater) in the presence of the candidate compound than in its absence, the candidate compound is identified as a stimulator of marker mRNA or protein expression. Conversely, when expression of marker mRNA or protein is less (statistically significantly less) in the presence of the candidate compound than in its absence, the candidate compound is identified as an inhibitor of marker mRNA or protein expression. The level of marker mRNA or protein expression in the cells can be determined by methods described herein for detecting marker mRNA or protein.

In another aspect, the invention pertains to a combination of two or more of the assays described herein. For example, a modulating agent can be identified using a cell-based or a cell free assay, and the ability of the agent to modulate the activity of a marker protein can be further confirmed in vivo, e.g., in a whole animal model for a neurological disease, disorder, or condition, cancer, cellular transformation and/or tumorigenesis. An animal model for neurological disease, disorder, or condition is described in, for example, Ding, H., et al. (2000) Neurosurgical Focus 8(4), the contents of which are expressly incorporated herein by reference. Additional animal based models of neurological disease, disorders and conditions are well known in the art and include, for example, those described in Weiss, W. A. and Banerjee, A. (2004) Semin Cancer Biol. 14(1):71-7; Hickey M A and Chesselet M F. (2003) Cytogenet Genome Res. 100(1-4):276-86; and Hafezparast M, et al (2002) Lancet Neurol. 1(4):215-24. Animal models described in, for example, Chin L. et al (1999) Nature 400(6743):468-72, may also be used in the methods of the invention.

This invention further pertains to novel agents identified by the above-described screening assays. Accordingly, it is within the scope of this invention to further use an agent identified as described herein in an appropriate animal model. For example, an agent identified as described herein (e.g., a marker modulating agent, a small molecule, an antisense marker nucleic acid molecule, a ribozyme, a marker-specific antibody, or fragment thereof, a marker protein, a marker nucleic acid molecule, an RNA interfering agent, e.g., an siRNA molecule targeting a marker of the invention, or a marker-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

### VIII. PHARMACEUTICAL COMPOSITIONS

The small molecules, peptides, peptoids, peptidomimetics, polypeptides, RNA interfering agents, e.g., siRNA molecules, antibodies, ribozymes, and antisense oligonucleotides (also referred to herein as “active compounds” or “compounds”) corresponding to a marker of the invention can be incorporated into pharmaceutical compositions suitable for administration. Such compositions typically comprise the small molecules, peptides, peptoids, peptidomimetics, polypeptides, RNA interfering agents, e.g., siRNA molecules, antibodies, ribozymes, or antisense oligonucleotides and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid corresponding to a marker of the invention. Such methods comprise formulating a pharmaceutically acceptable carrier with an agent which modulates expression or activity of a polypeptide or nucleic acid corresponding to a marker of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid corresponding to a marker of the invention and one or more additional active compounds.

It is understood that appropriate doses of small molecule agents and protein or polypeptide agents depends upon a number of factors within the knowledge of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid molecule or polypeptide of the invention. Small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram).

As defined herein, a therapeutically effective amount of an RNA interfering agent, e.g., siRNA, (i.e., an effective dosage) ranges from about 0.001 to 3,000 mg/kg body weight, preferably about 0.01 to 2500 mg/kg body weight, more preferably about 0.1 to 2000, about 0.1 to 1000 mg/kg body weight, 0.1 to 500 mg/kg body weight, 0.1 to 100 mg/kg body weight, 0.1 to 50 mg/kg body weight, 0.1 to 25 mg/kg body weight, and even more preferably about 1 to 10 mg/kg, 2 to 9 mg/kg, 3 to 8 mg/kg, 4 to 7 mg/kg, or 5 to 6 mg/kg body weight. Treatment of a subject with a therapeutically effective amount of an RNA interfering agent can include a single treatment or, preferably, can include a series of treatments. In a preferred example, a subject is treated with an RNA interfering agent in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks.

Exemplary doses of a protein or polypeptide include gram, milligram or microgram amounts per kilogram of subject or sample weight (e.g. about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the epithelium). A method for lipidation of antibodies is described by Cruikshank et al. (1997) J. Acquired Immune Deficiency Syndromes and Human Retrovirology 14:193.

The nucleic acid molecules corresponding to a marker of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The RNA interfering agents, e.g., siRNAs used in the methods of the invention can be inserted into vectors. These constructs can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the vector can include the RNA interfering agent, e.g., the siRNA vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### IX. PREDICTIVE MEDICINE

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining the amount, structure, and/or activity of polypeptides or nucleic acids corresponding to one or more markers of the invention, in order to determine whether an individual is at risk of developing a neurological disease, disorder, or condition. Such assays can be used for prognostic or predictive purposes to thereby prophylactically treat an individual prior to the onset of a neurological disease, disorder, or condition.

The present invention also provides methods of diagnosing tumor grade, e.g., glioma grade, clinical outcome, and prognosis for a subject afflicted with a tumor, e.g., a glioma. For example, the markers of the present invention may be used to determine whether a tumor, e.g., a glioma, is a high grade tumor or a low grade tumor, to predict the responsiveness of a tumor to certain treatment regimens, and to determine the prognosis of a subject with a tumor, e.g., a glioma.

Yet another aspect of the invention pertains to monitoring the influence of agents (e.g., drugs or other compounds administered either to inhibit a neurological disease, disorder, or condition, or to treat or prevent any other disorder {i.e. in order to understand any carcinogenic effects that such treatment may have}) on the amount, structure, and/or activity of a marker of the invention in clinical trials. These and other agents are described in further detail in the following sections.

A. Diagnostic Assays

1. Methods for Detection of Copy Number

Methods of evaluating the copy number of a particular marker or chromosomal region are well known to those of skill in the art. The presence or absence of chromosomal gain or loss can be evaluated simply by a determination of copy number of the regions or markers identified herein.

Methods for evaluating copy number of encoding nucleic acid in a sample include, but are not limited to, hybridization-based assays. For example, one method for evaluating the copy number of encoding nucleic acid in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid.

An alternative means for determining the copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

Preferred hybridization-based assays include, but are not limited to, traditional “direct probe” methods such as Southern blots or in situ hybridization (e.g., FISH), and “comparative probe” methods such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes are typically labeled, e.g., with radioisotopes or fluorescent reporters. Preferred probes are sufficiently long so as to specifically hybridize with the target nucleic acid(s) under stringent conditions. The preferred size range is from about 200 bases to about 1000 bases.

In some applications it is necessary to block the hybridization capacity of repetitive sequences. Thus, in some embodiments, tRNA, human genomic DNA, or Cot-I DNA is used to block non-specific hybridization.

In CGH methods, a first collection of nucleic acids (e.g. from a sample, e.g., a possible tumor) is labeled with a first label, while a second collection of nucleic acids (e.g. a control, e.g., from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number. Array-based CGH may also be performed with single-color labeling (as opposed to labeling the control and the possible tumor sample with two different dyes and mixing them prior to hybridization, which will yield ratio due to competitive hybridization to probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In Situ Hybridization Protocols, Choo, ed., Humana Press, Totowa, N.J. (1994), etc. In one embodiment, the hybridization protocol of Pinkel et al. (1998) Nature Genetics 20: 207-211, or of Kallioniemi (1992) Proc. Natl. Acad Sci USA 89:5321-5325 (1992) is used.

The methods of the invention are particularly well suited to array-based hybridization formats. Array-based CGH is described in U.S. Pat. No. 6,455,258, the contents of which are incorporated herein by reference.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and sybr green.

Other suitable amplification methods include, but are not limited to, ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

Loss of heterozygosity (LOH) mapping (Wang Z. C. et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93) may also be used to identify regions of amplification or deletion.

2. Methods for Detection of Gene Expression

Marker expression level can also be assayed as a method for diagnosis of cancer or risk for developing cancer. Expression of a marker of the invention may be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

Methods of detecting and/or quantifying the gene transcript (mRNA or cDNA made therefrom) using nucleic acid hybridization techniques are known to those of skill in the art (see Sambrook et al. supra). For example, one method for evaluating the presence, absence, or quantity of cDNA involves a Southern transfer as described above. Briefly, the mRNA is isolated (e.g. using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S, and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject, e.g., tumor cells. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated nucleic acid can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

The probes can be full length or less than the full length of the nucleic acid sequence encoding the protein. Shorter probes are empirically tested for specificity. Preferably nucleic acid probes are 20 bases or longer in length. (See, e.g., Sambrook et al. for methods of selecting nucleic acid probe sequences for use in nucleic acid hybridization.) Visualization of the hybridized portions allows the qualitative determination of the presence or absence of cDNA.

An alternative method for determining the level of a transcript corresponding to a marker of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-BetaReplicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Fluorogenic rtPCR may also be used in the methods of the invention. In fluorogenic rtPCR, quantitation is based on amount of fluorescence signals, e.g., TaqMan and sybr green. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers. For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a non-cancerous sample, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from cancer cells or normal cells of the same tissue type. The choice of the cell source is dependent on the use of the relative expression level. Using expression found in normal tissues as a mean expression score aids in validating whether the marker assayed is specific to the tissue from which the cell was derived (versus normal cells). In addition, as more data is accumulated, the mean expression value can be revised, providing improved relative expression values based on accumulated data. Expression data from normal cells provides a means for grading the severity of the cancer state.

In another preferred embodiment, expression of a marker is assessed by preparing genomic DNA or mRNA/cDNA (i.e. a transcribed polynucleotide) from cells in a subject sample, and by hybridizing the genomic DNA or mRNA/cDNA with a reference polynucleotide which is a complement of a polynucleotide comprising the marker, and fragments thereof. cDNA can, optionally, be amplified using any of a variety of polymerase chain reaction methods prior to hybridization with the reference polynucleotide. Expression of one or more markers can likewise be detected using quantitative PCR (QPCR) to assess the level of expression of the marker(s). Alternatively, any of the many known methods of detecting mutations or variants (e.g. single nucleotide polymorphisms, deletions, etc.) of a marker of the invention may be used to detect occurrence of a mutated marker in a subject.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a marker of the invention. If polynucleotides complementary to or homologous with are differentially detectable on the substrate (e.g. detectable using different chromophores or fluorophores, or fixed to different selected positions), then the levels of expression of a plurality of markers can be assessed simultaneously using a single substrate (e.g. a “gene chip” microarray of polynucleotides fixed at selected positions). When a method of assessing marker expression is used which involves hybridization of one nucleic acid with another, it is preferred that the hybridization be performed under stringent hybridization conditions.

In another embodiment, a combination of methods to assess the expression of a marker is utilized.

Because the compositions, kits, and methods of the invention rely on detection of a difference in expression levels or copy number of one or more markers of the invention, it is preferable that the level of expression or copy number of the marker is significantly greater than the minimum detection limit of the method used to assess expression or copy number in at least one of normal cells and cancerous cells.

3. Methods for Detection of Expressed Protein

The activity or level of a marker protein can also be detected and/or quantified by detecting or quantifying the expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like. A skilled artisan can readily adapt known protein/antibody detection methods for use in determining whether cells express a marker of the present invention.

A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In a preferred embodiment, the antibody is labeled, e.g. a radio-labeled, chromophore-labeled, fluorophore-labeled, or enzyme-labeled antibody). In another embodiment, an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a protein corresponding to the marker, such as the protein encoded by the open reading frame corresponding to the marker or such a protein which has undergone all or a portion of its normal post-translational modification, is used.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of a polypeptide in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind a polypeptide. The anti-polypeptide antibodies specifically bind to the polypeptide on the solid support. These antibodies may be directly labeled or alternatively may be subsequently detected using labeled antibodies (e.g., labeled sheep anti-mouse antibodies) that specifically bind to the anti-polypeptide.

In a more preferred embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In a preferred embodiment, the capture agent is an antibody that specifically binds a polypeptide. The antibody (anti-peptide) may be produced by any of a number of means well known to those of skill in the art.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled anti-antibody. Alternatively, the labeling agent may be a third moiety, such as another antibody, that specifically binds to the antibody/polypeptide complex.

In one preferred embodiment, the labeling agent is a second human antibody bearing a label. Alternatively, the second antibody may lack a label, but it may, in turn, be bound by a labeled third antibody specific to antibodies of the species from which the second antibody is derived. The second can be modified with a detectable moiety, e.g. as biotin, to which a third labeled molecule can specifically bind, such as enzyme-labeled streptavidin.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

As indicated above, immunoassays for the detection and/or quantification of a polypeptide can take a wide variety of formats well known to those of skill in the art.

Preferred immunoassays for detecting a polypeptide are either competitive or noncompetitive. Noncompetitive immunoassays are assays in which the amount of captured analyte is directly measured. In one preferred “sandwich” assay, for example, the capture agent (anti-peptide antibodies) can be bound directly to a solid substrate where they are immobilized. These immobilized antibodies then capture polypeptide present in the test sample. The polypeptide thus immobilized is then bound by a labeling agent, such as a second human antibody bearing a label.

In competitive assays, the amount of analyte (polypeptide) present in the sample is measured indirectly by measuring the amount of an added (exogenous) analyte (polypeptide) displaced (or competed away) from a capture agent (anti peptide antibody) by the analyte present in the sample. In one competitive assay, a known amount of, in this case, a polypeptide is added to the sample and the sample is then contacted with a capture agent. The amount of polypeptide bound to the antibody is inversely proportional to the concentration of polypeptide present in the sample.

In one particularly preferred embodiment, the antibody is immobilized on a solid substrate. The amount of polypeptide bound to the antibody may be determined either by measuring the amount of polypeptide present in a polypeptide/antibody complex, or alternatively by measuring the amount of remaining uncomplexed polypeptide. The amount of polypeptide may be detected by providing a labeled polypeptide.

The assays of this invention are scored (as positive or negative or quantity of polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of polypeptide.

Antibodies for use in the various immunoassays described herein, can be produced as described below.

In another embodiment, level (activity) is assayed by measuring the enzymatic activity of the gene product. Methods of assaying the activity of an enzyme are well known to those of skill in the art.

In vivo techniques for detection of a biomarker protein include introducing into a subject a labeled antibody directed against the protein. For example, the antibody can be labeled with a radioactive marker whose presence and location in a subject can be detected by standard imaging techniques.

Certain markers identified by the methods of the invention may be secreted proteins. It is a simple matter for the skilled artisan to determine whether any particular marker protein is a secreted protein. In order to make this determination, the marker protein is expressed in, for example, a mammalian cell, preferably a human cell line, extracellular fluid is collected, and the presence or absence of the protein in the extracellular fluid is assessed (e.g. using a labeled antibody which binds specifically with the protein).

The following is an example of a method which can be used to detect secretion of a protein. About 8×10⁵ 293T cells are incubated at 37° C. in wells containing growth medium (Dulbecco's modified Eagle's medium {DMEM} supplemented with 10% fetal bovine serum) under a 5% (v/v) CO2, 95% air atmosphere to about 60-70% confluence. The cells are then transfected using a standard transfection mixture comprising 2 micrograms of DNA comprising an expression vector encoding the protein and 10 microliters of LipofectAMINE™ (GIBCO/BRL Catalog no. 18342-012) per well. The transfection mixture is maintained for about 5 hours, and then replaced with fresh growth medium and maintained in an air atmosphere. Each well is gently rinsed twice with DMEM which does not contain methionine or cysteine (DMEM-MC; ICN Catalog no. 16-424-54). About 1 milliliter of DMEM-MC and about 50 microcuries of Trans-³⁵S™ reagent (ICN Catalog no. 51006) are added to each well. The wells are maintained under the 5% CO₂ atmosphere described above and incubated at 37° C. for a selected period. Following incubation, 150 microliters of conditioned medium is removed and centrifuged to remove floating cells and debris. The presence of the protein in the supernatant is an indication that the protein is secreted.

It will be appreciated that subject samples, e.g., a sample containing tissue or cells, e.g., neuroglial tissue or cells, e.g., astrocytes, whole blood, serum, plasma, buccal scrape, saliva, spinal fluid, cerebrospinal fluid, urine, stool, may contain cells therein, particularly when the cells are cancerous, and, more particularly, when the cancer is metastasizing, and thus may be used in the methods of the present invention. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the level of expression of the marker in the sample. Thus, the compositions, kits, and methods of the invention can be used to detect expression of markers corresponding to proteins having at least one portion which is displayed on the surface of cells which express it. It is a simple matter for the skilled artisan to determine whether the protein corresponding to any particular marker comprises a cell-surface protein. For example, immunological methods may be used to detect such proteins on whole cells, or well known computer-based sequence analysis methods (e.g. the SIGNALP program; Nielsen et al., 1997, Protein Engineering 10:1-6) may be used to predict the presence of at least one extracellular domain (i.e. including both secreted proteins and proteins having at least one cell-surface domain). Expression of a marker corresponding to a protein having at least one portion which is displayed on the surface of a cell which expresses it may be detected without necessarily lysing the cell (e.g. using a labeled antibody which binds specifically with a cell-surface domain of the protein).

The invention also encompasses kits for detecting the presence of a polypeptide or nucleic acid corresponding to a marker of the invention in a biological sample, e.g., a sample containing tissue or cells, e.g., neuroglial tissue or cells, e.g., astrocytes, whole blood, serum, plasma, buccal scrape, saliva, spinal fluid, cerebrospinal fluid, urine, stool. Such kits can be used to determine if a subject is suffering from or is at increased risk of developing cancer. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

For antibody-based kits, the kit can comprise, for example: (1) a first antibody (e.g., attached to a solid support) which binds to a polypeptide corresponding to a marker of the invention; and, optionally, (2) a second, different antibody which binds to either the polypeptide or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

4. Method for Detecting Structural Alterations

The invention also provides a method for assessing whether a subject is afflicted with cancer or is at risk for developing cancer by comparing the structural alterations, e.g., mutations or allelic variants, of a marker in a cancer sample with the structural alterations, e.g., mutations of a marker in a normal, e.g., control sample. The presence of a structural alteration, e.g., mutation or allelic variant in the marker in the cancer sample is an indication that the subject is afflicted with cancer.

A preferred detection method is allele specific hybridization using probes overlapping the polymorphic site and having about 5, 10, 20, 25, or 30 nucleotides around the polymorphic region. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix™). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism in the 5′ upstream regulatory element can be determined in a single hybridization experiment.

In other detection methods, it is necessary to first amplify at least a portion of a marker prior to identifying the allelic variant. Amplification can be performed, e.g., by PCR and/or LCR (see Wu and Wallace (1989) Genomics 4:560), according to methods known in the art. In one embodiment, genomic DNA of a cell is exposed to two PCR primers and amplification for a number of cycles sufficient to produce the required amount of amplified DNA. In preferred embodiments, the primers are located between 150 and 350 base pairs apart.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al., (1988) Bio/Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the alt can be used to directly sequence at least a portion of a marker and detect allelic variants, e.g., mutations, by comparing the sequence of the sample sequence with the corresponding reference (control) sequence. Exemplary sequencing reactions include those based on techniques developed by Maxam and Gilbert (Proc. Natl. Acad Sci USA (1977) 74:560) or Sanger (Sanger et al. (1977) Proc. Nat. Acad. Sci. 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Köster; U.S. Pat. No. 5,547,835 and international patent application Publication Number WO 94/21822 entitled “DNA Sequencing by Mass Spectrometry Via Exonuclease Degradation” by H. Köster), and U.S. Pat. No. 5,605,798 and International Patent Application No. PCT/US96/03651 entitled DNA Diagnostics Based on A Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing.”

In some cases, the presence of a specific allele of a marker in DNA from a subject can be shown by restriction enzyme analysis. For example, a specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of a marker allelic variant with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al (1988) Proc. Natl. Acad Sci USA 85:4397; Saleeba et al (1992) Methods Enzymol. 217:286-295. In a preferred embodiment, the control or sample nucleic acid is labeled for detection.

In another embodiment, an allelic variant can be identified by denaturing high-performance liquid chromatography (DHPLC) (Oefner and Underhill, (1995) Am. J. Human Gen. 57:Suppl. A266). DHPLC uses reverse-phase ion-pairing chromatography to detect the heteroduplexes that are generated during amplification of PCR fragments from individuals who are heterozygous at a particular nucleotide locus within that fragment (Oefher and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility is used to identify the type of marker allelic variant. For example, single strand conformation polymorphism (SSCP) may be used to detect differences in electrophoretic mobility between mutant and wild type nucleic acids (Orita et al (1989) Proc Natl. Acad. Sci. USA 86:2766, see also Cotton (1993) Mutat Res 285:125-144; and Hayashi (1992) Genet Anal Tech Appl 9:73-79).

Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al, (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of marker. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

The invention further provides methods for detecting single nucleotide polymorphisms in a marker. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Several primer-guided nucleotide incorporation procedures for assaying polymorphic sites in DNA have been described (Komher, J. S. et al., (1989) Nucl. Acids. Res. 17:7779-7784; Sokolov, B. P., (1990) Nucl. Acids Res. 18:3671; Syvanen, A.-C., et al., (1990) Genomics 8:684-692; Kuppuswamy, M. N. et al., (1991) Proc. Natl. Acad. Sci. (U.S.A.) 88:1143-1147; Prezant, T. R. et al., (1992) Hum. Mutat. 1:159-164; Ugozzoli, L. et al., (1992) GATA 9:107-112; Nyren, P. (1993) et al., Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. C., et al., (1993) Amer. J. Hum. Genet. 52:46-59).

For determining the identity of the allelic variant of a polymorphic region located in the coding region of a marker, yet other methods than those described above can be used. For example, identification of an allelic variant which encodes a mutated marker can be performed by using an antibody specifically recognizing the mutant protein in, e.g., immunohistochemistry or immunoprecipitation. Antibodies to wild-type marker or mutated forms of markers can be prepared according to methods known in the art.

Alternatively, one can also measure an activity of a marker, such as binding to a marker ligand. Binding assays are known in the art and involve, e.g., obtaining cells from a subject, and performing binding experiments with a labeled ligand, to determine whether binding to the mutated form of the protein differs from binding to the wild-type of the protein.

B. Pharmacogenomics

Agents or modulators which have a stimulatory or inhibitory effect on amount and/or activity of a marker of the invention can be administered to individuals to treat (prophylactically or therapeutically) a neurological disease, disorder, or condition in the subject. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the amount, structure, and/or activity of the invention in an individual can be determined to thereby select appropriate agent(s) for therapeutic or prophylactic treatment of the individual.

Pharmacogenomics deals with clinically significant variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the amount, structure, and/or activity of a marker of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of amount, structure, and/or activity of a marker of the invention.

C. Monitoring Clinical Trials

Monitoring the influence of agents (e.g., drug compounds) on amount, structure, and/or activity of a marker of the invention can be applied not only in basic drug screening, but also in clinical trials. For example, the effectiveness of an agent to affect marker amount, structure, and/or activity can be monitored in clinical trials of subjects receiving treatment for a neurological disease, disorder, or condition. In a preferred embodiment, the present invention provides a method for monitoring the effectiveness of treatment of a subject with an agent (e.g., an agonist, antagonist, peptidomimetic, protein, peptide, antibody, nucleic acid, antisense nucleic acid, ribozyme, small molecule, RNA interfering agent, or other drug candidate) comprising the steps of (i) obtaining a pre-administration sample from a subject prior to administration of the agent; (ii) detecting the amount, structure, and/or activity of one or more selected markers of the invention in the pre-administration sample; (iii) obtaining one or more post-administration samples from the subject; (iv) detecting the amount, structure, and/or activity of the marker(s) in the post-administration samples; (v) comparing the level of expression of the marker(s) in the pre-administration sample with the amount, structure, and/or activity of the marker(s) in the post-administration sample or samples; and (vi) altering the administration of the agent to the subject accordingly. For example, increased administration of the agent can be desirable to increase amount and/or activity of the marker(s) to higher levels than detected, i.e., to increase the effectiveness of the agent. Alternatively, decreased administration of the agent can be desirable to decrease amount and/or activity of the marker(s) to lower levels than detected, i.e., to decrease the effectiveness of the agent.

This invention is further illustrated by the following examples which should not be construed as limiting. The contents of all references, figures, tables, Appendices, Accession Numbers, patents and published patent applications cited throughout this application are hereby incorporated by reference.

### EXEMPLIFICATION

### Example 1

### Identification of Novel Astrocyte-Specific Markers

Astrocytes have long been postulated to play a role in normal brain function by regulating neurotransmitters (glutamate), ions (K+) and metabolic substrates (glucose), as well as respond to brain injuries caused by trauma, stroke and seizures. More recently its becoming increasingly clear that astrocytes may also play an important role(s) in neurodegenerative diseases (Alzheimers). Astrocytes or their precursor also is the likely cell of origin for the most frequent and the deadliest form of primary brain tumor, Glioblastoma multiforme. While the distinction between neuronal stem cells and astrocytes is unclear, evidence suggests that a distinct subtype of astrocytes in the subventricular zone (SVZ) and in the subgranular zone of the hippocampus, may retain the ability to differentiate into neurons, oligodendrocytes and astrocytes, and can functionally be defined as a stem cell. The potential importance of this question to both basic and clinical research is clear and the goal of identifying markers which are unique to astrocytes of the outmost importance.

### A. Materials and Methods

NSC and astrocyte culture techniques. Primary neural stem cells (NSCs) were isolated from murine E13.5 embryos as described (Reynolds, B. A. & Weiss, S. (1992) Science 255, 1707-1710). Neurospheres were differentiated into astrocyte predominant cultures by exposure to: 10% fetal bovine serum, CNTF (50 ng/ml), BMP2 (50 ng/ml) or PACAP (50 ng/ml). Neuronal cultures were derived from E13.5 hippocampal primordia by previously described methods. Primary cortical astrocytes were isolated from 1-2 day old neonates and prepared according to published methods (McCarthy, K. D. & de Vellis, J. (1980) J Cell Biol 85, 890-902).

The transcriptional profile strategy for the identification of astrocyte-relevant transcripts exploited various primary and induced astrocyte culture systems, primary neuron cultures, NSCs, and regionally defined and developmentally staged CNS tissues including E13.5 cortex, the corpus callosum isolated from postnatal (P5 and P10) brain, and neocortical layers II-VI and subpial/glial limitans of the 6 week old adult brain. Total RNA was used to prepare in vitro transcribed amplified probes and the hybridized to oligonucleotide microarrays (Affymetrix U74Av2) representing 12488 probe sets representing 8,585 mouse genes.

Data processing and normalization. The CEL files were obtained using Affymetrix Microarray Suite software. The DNA-Chip Analyzer (dChip.org, version 1.3) was used to normalize all CEL files to the baseline arrays and compute the model-based expression (PM-only model) (Li, C. a. W., W. H. (2003) in The analysis of gene expression data: methods and software, ed. Parmigiani, G., Irizarry, E. S. G. R., Zeger, S. L. (Springer)). Arrays were normalized within tissue types by picking the baseline arrays for each tissue type. When calculating the model-based expression, constant array outliers within each of the six groups—10% FCS, CNTF/BMP2/PACAP, hippocampal neurons, primary cortical astrocytes, the gray matter and the corpus callosum/glial limitans were considered as a real biological effect.

Gene annotation. Each probe set was mapped to a LocusLink ID using the annotation provided by Affymetrix. LocusLink IDs and their annotations were updated by the most recent NCBI LocusLink database annotations.

Pooling the replicates and lower bound fold change. Replicate samples were averaged by considering model-based standard errors of individual expression values using a resampling method (Li, C. a. W., W. H. (2003) in The analysis of gene expression data: methods and software, ed. Parmigiani, G., Irizarry, E. S. G. R., Zeger, S. L. (Springer)). To identify the genes with large change between two groups of replicates, the parametric estimation of lower bound fold change (LBFC) was used as the measure of change (Li, C. & Wong, W. H. (2001) Proc Natl Acad Sci USA 98, 31-6). The selection criteria of selecting genes with LBFC of equal to or greater than 3 typically corresponds to a fold change of at least 5 in conventional gene expression assays such as quantitative PCR.

Selection of genes that contribute most in distinguishing the astrocyte culture samples and NSC/early embryo brain samples. 2,061 genes with large variation and high presence call (by dChip) were chosen and R-SVM was used to identify the genes contributing most in distinguishing two sample groups (Zhang, X. a. W., W. H. (2001) Technical Report, Department of Biostatistics, Harvard University). Unlike other gene-wise analysis, this algorithm finds a set of genes that work as a group in separating two sample groups by recursively building linear Support Vector Machines (SVM) (Collobert, R., and Bengio, S. (2001) Journal of Machine Learning Research 1, 143-160). To overcome possible overfitting, the leave-one-out cross-validation was performed and the error rate was zero. Both the gene selection step and the SVM building step were included in cross-validation.

Selection of up-regulated genes in each sample type. A gene was determined to be up regulated in a sample type if (1) the gene is called present (by dChip) 50% or higher in at least one of two groups being compared, and (2) its LBFC is greater than the 3.

Sample clustering and gene clustering. For each clustering, genes with sufficient variation and high presence call (by dChip) percentage were selected. dChip performed the hierarchical clustering using agglomerative method (bottom up) with 1 minus correlation as the distance between genes and centroid-linkage as the inter-cluster distance. A sequential clustering algorithm (developed by Tseng and Wong, 2003, Harvard Biostatistics Technical report and software) was also used to look for the genes that ‘tightly’ co-regulate with in-situ validated astrocyte specific genes. In contrast to conventional hierarchical clustering, which clusters all genes, this algorithm finds the tightest clusters sequentially and exclude genes with uncertain memberships to the clusters.

Finding significant functional categories in a gene list with size n. The annotation information in the dChip MG_U74Av2 gene information file was also used to classify genes in a list into different functional categories of gene ontology and to compute their significance values (p<0.02).

Validation of Astrocyte Candidate Genes by In-Situ hybridization. Probes were scored for labeling efficacy, CNS expression, and brain region/cell type distribution. Genes with a “glial” expression pattern across neural development (i.e. increasing expression from E13.5 to adult, expression in white matter, glial limitans, SVZ) were in most cases easily separated from those with a neuronal only type pattern (i.e. hippocampus, specific cortical layers) by anatomical assessment alone. Genes suspected to be ‘astrocytic/glial’ by anatomic criteria were stringently validated for cell type specificity by combining ISH with several lineage specific immunohistochemical markers, including GFAP (astrocytic), Olig2/(oligodendroglial), or NeuN (neuronal).

### B. Results

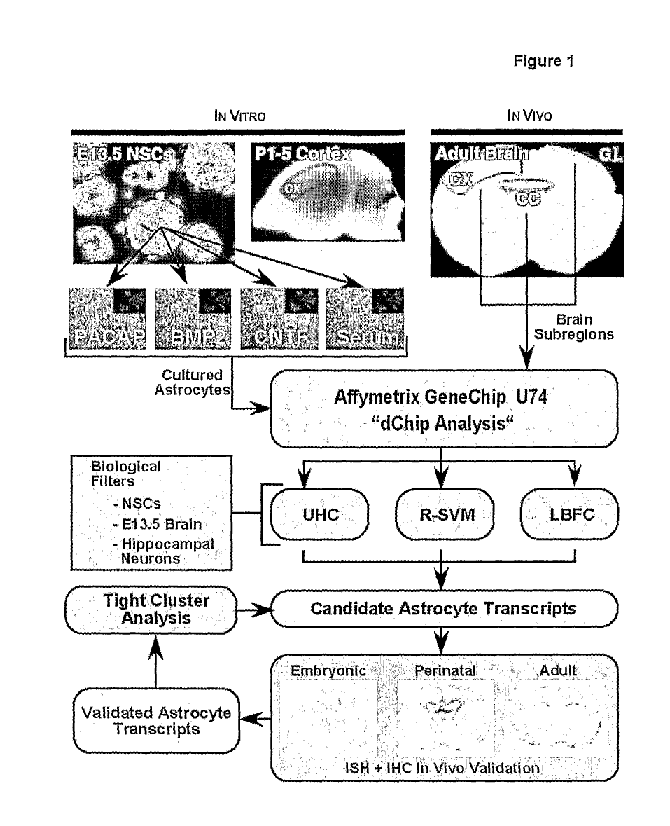

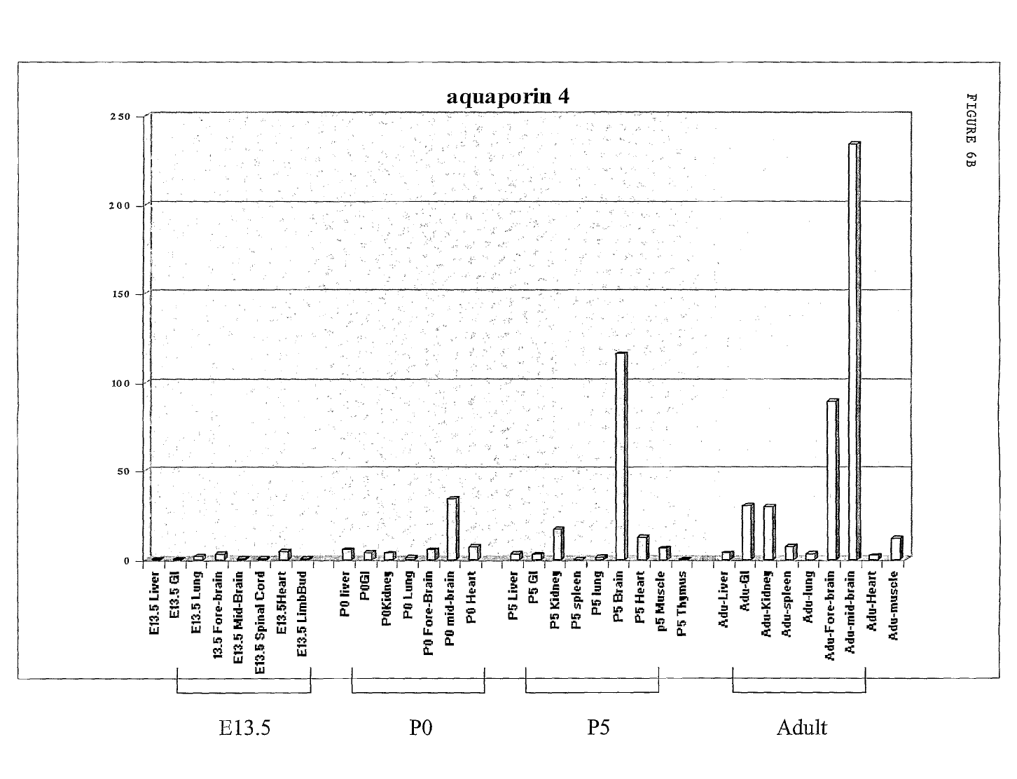

Reproducibility and Distinctiveness of Transcriptional Profiles. To identify astrocyte-specific genes, a multilevel biological prioritization and filtering scheme was implemented based upon the combined use of several in vitro astrocyte differentiation systems, isolated primary astrocytes from the perinatal brain, and various microdissected astrocyte-rich regions of the telencephalon (FIG. 1). The first series of experiments exploited the capacity of various agents (10% FCS, CNTF, BMP2, or PACAP) to elicit a common astrocytic phenotype from NSCs, reasoning that comparative transcriptional profiles across these protocols should enrich for genes common to most astrocytes regardless of isolation and induction procedures. Replicates within a given experimental modality or tissue type demonstrated a high degree of reproducibility (correlation coefficient 0.95-0.99) and when analyzed as groups, highlighted the distinctiveness of the astrocyte profile from the profiles of neurons, NSCs and embryonic cortex (FIG. 5). The reproducibility and fidelity of these microarray datasets serve to document the quality and purity of the samples used, thereby supporting their use in molecularly defining the astrocyte lineage.

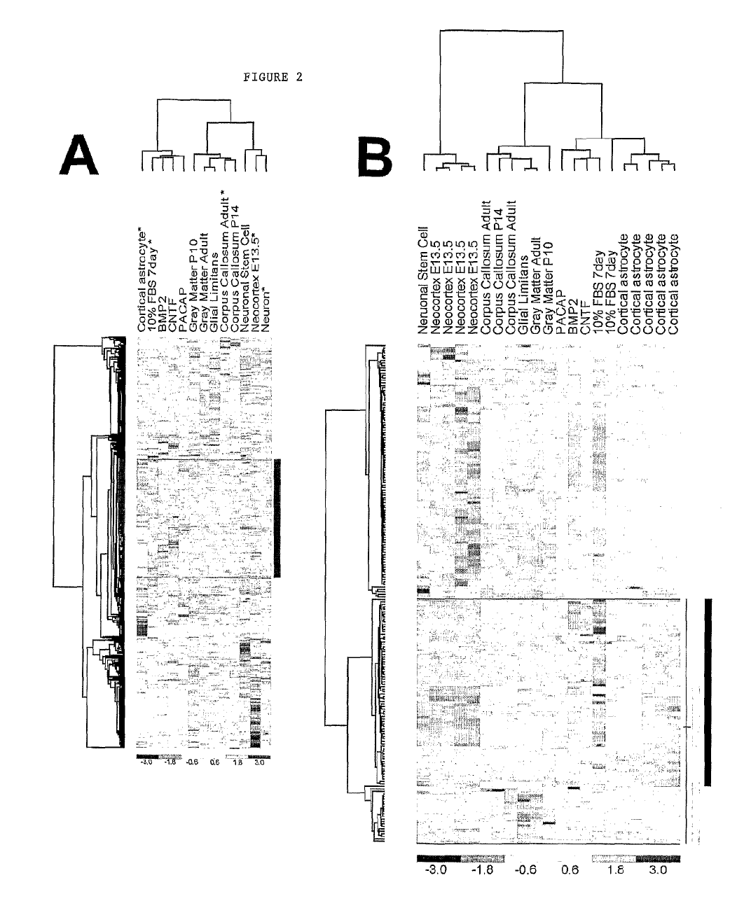

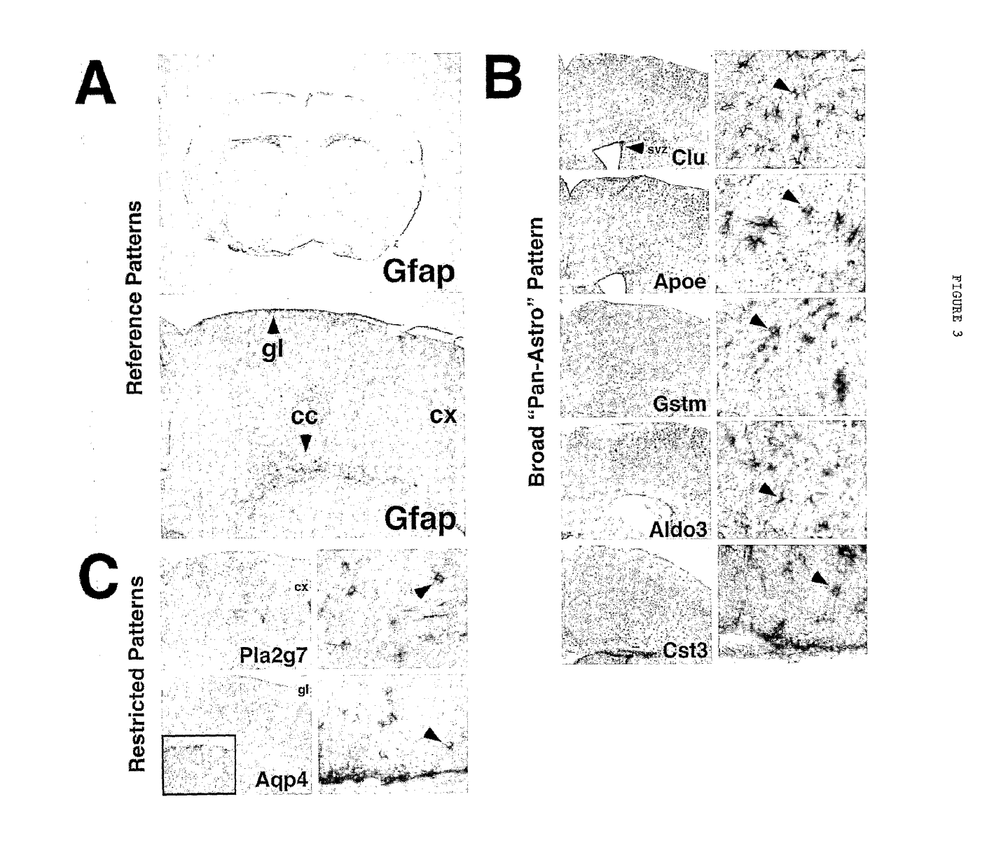

Identification of Novel Astrocyte Markers. To determine that all astrocytes share a common transcriptional profile, unsupervised hierarchical clustering (UHC) of all experimental samples was performed (FIG. 2A). This unbiased approach organized the experimental samples into two major groups, one consisting of multipotent NSC and lineage committed progenitors (NSCs, E13.5 neocortex, and hippocampal neuroblasts) and the other containing differentiated astrocytes. The remarkably similar expression profiles among the various in vitro differentiated astrocytes and their tight association with the primary cortical astrocytes indicates that, despite the distinct signaling pathways engaged by the various agents used to drive commitment to the astrocyte lineage, they share a similar molecular profile. Similarly, the tight clustering of microdissected brain subregions, corpus callosum, gray matter and glial limitans, demonstrates a high degree of transcriptional relatedness that is related in part to their astrocyte-rich composition. The lack of significant overlap among the in vitro and in vivo UHC clusters likely results from a combination of the cell type heterogeneity of the CNS tissues as well as culture-induced stimulation of astrocytes to assume a ‘reactive’ rather than the resting state typical of astrocytes in the normal brain. Together, these findings support the use of the multiple sample types as an experimental approach for the identification of candidate astrocyte genes that are expressed in normal brain.

An in-depth bioinformatic search for astrocyte-specific genes comprised three different methods: (i) a biased search for genes with an expression pattern similar to the best known astrocyte marker, GFAP, (ii) an unbiased search by a novel class prediction tool, Recursive-Supervised Machine (R-SVM) analysis, and (iii) an empirical threshold approach to identify a common set of genes among complementary data sets. In the GFAP cluster analysis, GFAP captured a group of 393 genes that were differentially expressed by all in vitro astrocyte samples (FIG. 2A). While this cluster likely represents a potential source of novel astrocyte genes (see Table 2 for complete list)), it is notable that no clear GFAP subcluster emerged and, correspondingly, a random sampling of genes among the GFAP cluster revealed their limited expression in CNS astrocytes by RNA ISH (see below). All astrocyte candidate genes identified by R-SVM, ‘common in-vitro’ and ‘common in-vivo’. Next, to avoid the bias of so-called signature genes, R-SVM analysis was employed to identify genes that ‘as a group’ (unlike genes found by other traditional two-group comparison methods) contribute most to distinguishing the two groups (Zhang, X. a. W., W. H. (2001) Technical Report, Department of Biostatistics, Harvard University). From a total of 2,005 genes, which show sufficient variation in expression over all samples, R-SVM identified a set of 85 genes that most significantly contribute to the astrocyte group (FIG. 2B). The relative contribution of each gene in distinguishing astrocytes from the NSC and early-lineage committed cells is presented in Table 3. Table 3 shows that the union of these 3 experimental datasets produced a list of 153 genes. For R-SVM, the relative contribution of each gene to distinguishing the astrocyte group is presented as percent contribution of the total gene pool contribution. Also presented for direct comparison is the maximum LBFC of genes from the common in-vitro and common in-vivo datasets. There is considerable concordance among the datasets; genes which contribute most to distinguishing the astrocyte group R-SVM, also have a high LBF change value. Furthermore it shows that once the R-SVM list is exhausted (R-SVM contribution=0) there are still a large number of astrocyte candidate genes, which are identified either by the common in-vitro or common in-vivo lists, or both. These data support the usefulness of these complimentary datasets. Importantly, the gene that contributes most significantly is the main lipid transport protein in the CNS, apolipoprotein E (apoE) (8.8%), whereas the GFAP is ranked number 22 (0.43%).

To capture a greater representation of the astrocyte transcriptome in a physiological context, empirical threshold studies were also conducted to compare the datasets of corpus callosum, gray matter and glial limitans with those derived from E13.5 cortex (a period of development preceding the birth of astrocytes, circa E17.5). The application of the expression criteria of >3 LBFC relative to E13.5 cortex yielded 84 genes in the corpus callosum, 103 in the gray matter and 100 in glial limitans. An intersection of these three gene lists generated a list of 47 ‘common in vivo’ genes, which could represent candidate astrocyte genes since this cell type is common to these brain subregions. Of these 47 ‘common in-vivo’ genes, 8 genes were differentially expressed (>3 LBFC) among the ‘common in vitro’ genes which are defined as genes expressed in at least 4 of 5 in vitro astrocyte datasets (a complete list of the union of these genes is presented in Table 3. These data support the assumption that there may be qualitative and quantitative differences between the genes expressed by astrocytes maintained in cell culture and those found in the normal brain. It is also possible that this limited overlap reflects regionally restricted expression patterns of astrocytes in various brain microenvironments (i.e., only in corpus callosum, gray matter, or glial limitans). To obtain a more complete view of potential molecular diversity of astrocytes in the adult brain, a pair-wise comparison of each CC, GM and GL gene list was performed and union of the four in vitro astrocyte datasets. These comparisons yielded 33, 29 and 23 genes for the CC, GM and GL, respectively (see Tables 4A, 4B, and 4C. Table 4A shows that 33/41 genes/probesets were up-regulated in corpus callosum and at least one culture system. Table 4B shows that 29/37 genes/probesets were up-regulated in gray matter and at least one culture system. Table 4C shows that 23/30 genes/probe-sets were up-regulated in glial limitans and at least one culture system.

These data underscore that markedly distinct molecular profiles can emerge through the use of specific tissues, model systems and experimental conditions and the application of specific bioinformatic methods, further justifying the comprehensive set of comparisons, bioinformatic approaches, model systems and distinct astrocyte and non-astrocytic tissues and cell types.

Validation of candidate astrocyte-specific genes. A combination of ISH and lineage-specific immunohistochemical (IHC) markers were utilized to assess the temporal and spatial patterns of a cross-section of the candidate astrocyte-associated genes derived from various datasets.

Of the 83 genes that were differentially expressed (>3 LBFC) by exposure to 10% FCS, the top 19 genes were tested by ISH, of these, 2 (GFAP and aquaporin 4) identified astrocytes in adult mouse brain (Table 1). The ability to identify astrocytes by ISH improved moderately when genes were selected from among the large UHC of astrocyte associated genes (so-called, GFAP co-cluster) (19 of 37). The R-SVM approach proved to be most effective with 10 of 13 candidate genes identifying astrocytes, while the ‘common in vivo ’ and ‘common in vitro’ lists identified and 9 of 12 genes and 8 of 13 genes, respectively.

Table 1 summarizes the validation of astrocyte candidate genes by combination of ISH and IHC. The Table shows that a random selection of differentially expressed (>3LBFC) genes from in vitro differentiated NSC's produces poor prediction of astrocyte specific genes. This performance improves among a common set of genes differentially expressed following NSC differentiation by serum, CNTF, BMP2, PACAP (UHC GFAP co-cluster). Even greater success was achieved with R-SVM, ‘common in vitro’ and ‘common in vivo’ data sets in validating astrocyte candidate genes by combined ISH and IHC.

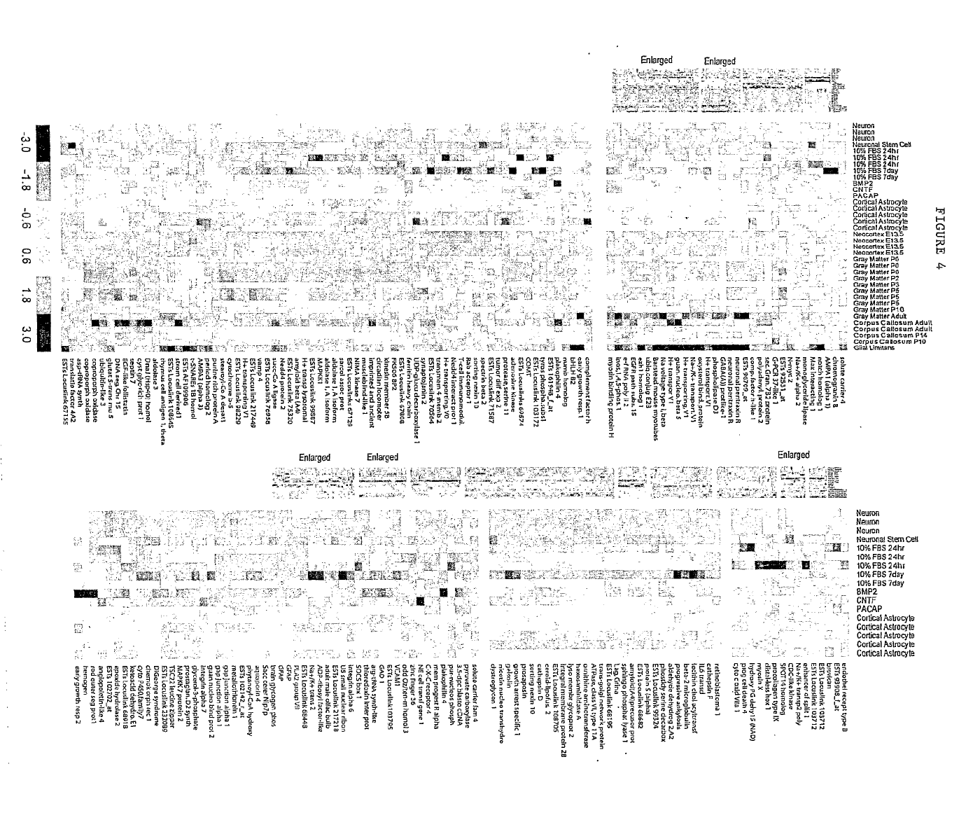

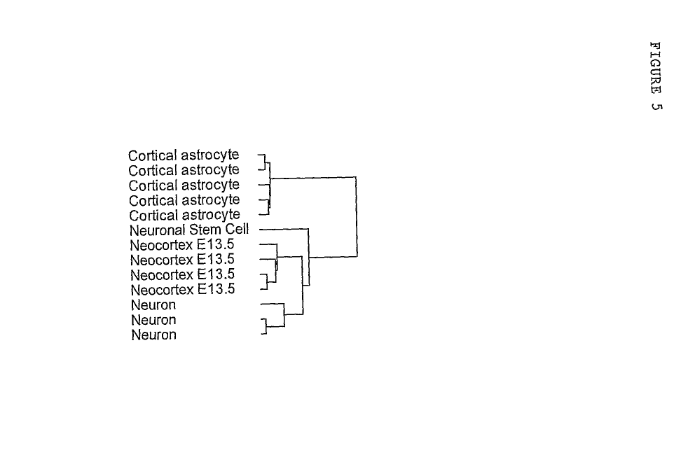

Notably, the validated astrocyte genes exhibit widely varying patterns of expression with only a small subset showing co-expression with GFAP, yet staining cells with unequivocal astrocyte morphology. Of the genes with a restricted expression pattern, 3 genes had a GFAP-like pattern with cells predominantly labeled in the white matter, GL, and SVZ (aquaporin 4, brain glycogen phosphorylase, and brevican). Aquaporin 4 was predominantly expressed by astrocytes of the GL in the subpial and perivascular locations, as reported previously (Nielsen, S., et al. (1997) J Neurosci 17, 171-80), while 5 genes were prominently expressed in the SVZ and to a more limited extent in the adjacent gray matter (Id3, vascular cell adhesion molecule 1, N-myc downregulated 2, integral membrane protein 1, and endothelial differentiation receptor 1). In addition, 2 genes labeled ependymal cells (diazepam binding inhibitor and interleukin 6 signal transducer), these genes were included here since ependymal cells label positively with GFAP and may arise from a common precursor. Many genes had a heterogeneous expression pattern, labeling scattered populations of cells in the gray matter, while 5 genes had a broad pattern of expression labeling cells in the subventricular zone, white matter and throughout the gray matter of the telencephalon; these included clusterin, cystatin C, apoE, glutathione S-transferase and aldolase 3 (see FIG. 3; all validated genes are listed in Table 5 and Table 7).

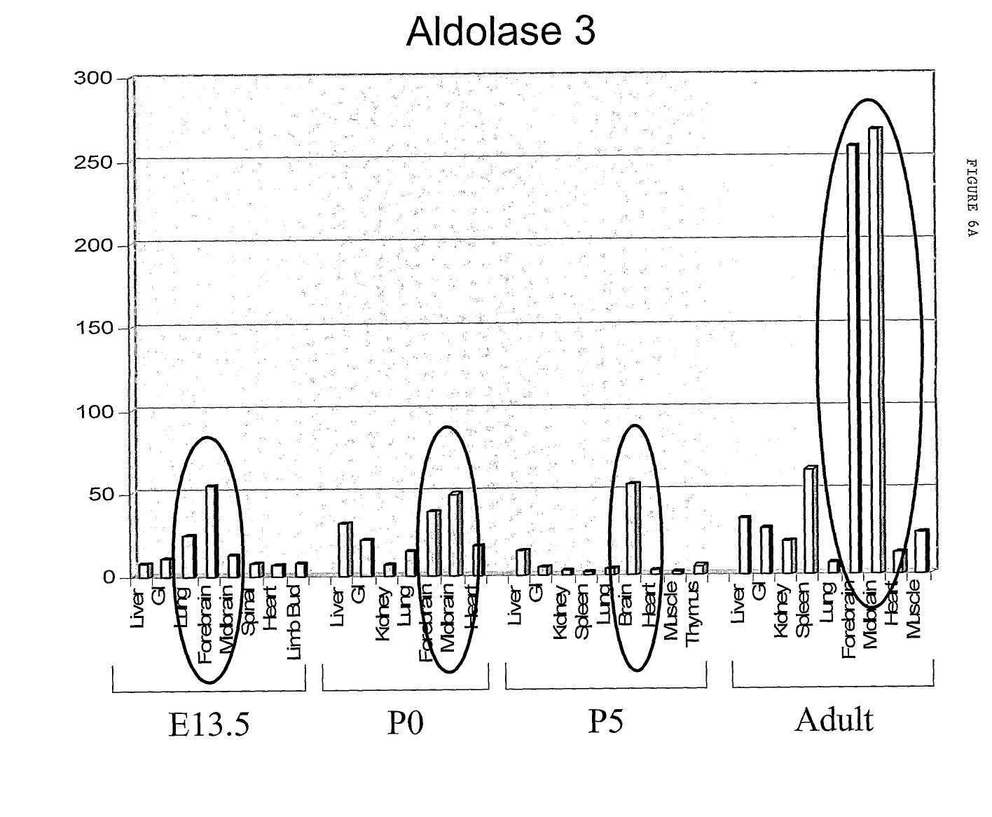

Tight Cluster Analysis of Candidate Astrocyte-Specific Genes. The collection of validated astrocyte genes makes possible an effective prospective bioinformatic identification of additional astrocyte genes and a more comprehensive molecular view of this lineage. To that end, a novel clustering algorithm capable of identifying genes that ‘tightly cluster’ with validated genes was used. 2,061 genes with sufficient variability over samples were selected for tight cluster analysis using both in vitro and in vivo samples, 51% of the probe sets were assigned to the top 30 tightest clusters of which 4 were identified by inclusion of 6 validated astrocyte genes (see FIG. 4A). These tight clusters are remarkable for identifying differentially expressed genes in both the cultured astrocytes and normal brain, but not in NSCs, neurons or embryonic brain. For the in vitro sample datasets, there were 9 tight clusters all of which contained one or more of our validated astrocyte-associated genes. Data presented in FIG. 4B shows 3 representatives of 9 tight clusters from the cultured astrocyte dataset, each identified by several ISH validated genes (total 16 in situ validated genes among the 9 tight clusters). Each tight cluster was dominated by a single, statistically significant (p<0.01), functional gene ontology category (see below and Table 6). Table 6 shows the functional categories of ISH validated astrocyte genes and the genes identified by tight cluster analysis, (the clusters and gene names are shown in FIG. 4; ‘T’, ‘M’, and ‘B’, represent top, middle and bottom tight clusters, respectively). Each tight cluster is represented by only one or two statistically significant functional categories. These data indicate that co-expression may predict a common function. Furthermore, these results suggest that tight cluster analysis is not only an efficient means of identifying cell type specific genes, it can also identify functionally related astrocyte genes, which reflect on normal astrocyte functions.

Among the 46 GFAP co-clustered genes identified by tight cluster algorithm (FIG. 4B), which exclude genes with uncertain membership, 5 of 7 genes tested by ISH proved to be astrocyte-specific (phospholipase A2 group 7, gap-junction channel protein 1-alpha, aquaporin 4, vascular cell adhesion molecule 1 and brain glycogen phosphorylase). These results underscore that the tight cluster tool represents an efficient means of identifying additional astrocyte markers. Furthermore, the performance of the tight cluster results continues to improve as more candidate genes are validated by ISH and that information is used to refine the tight clustering parameters.

Functional Annotation of Validated Astrocyte Genes and Tight Cluster Genes. Functional classification by the Gene Ontology (GO) database yielded many significant and distinct categories (see Table 6). Among them are categories consistent with known astrocyte function including genes encoding potent antioxidant activity (e.g., glutathione S-transferase, peroxiredoxin 5), excitatory amino acid uptake (solute carrier 1), immune modulation/chemotaxis (e.g., CXCR4, CX3-C motif 1), microvascular regulation (PLA2g7, thrombospondin, vascular cell adhesion molecule) and blood brain barrier function (aquaporin 4). Of special note is a prominently represented category linked to lipid transport and metabolism (e.g., apoE, fatty acid binding protein, steroyl CoA desaturase-2), providing evidence for the concept that astrocytes play an obligatory role in cholesterol synthesis and transport to neurons.

### Example 2

### Generation of a Mouse Model of Neuroblastoma

The generation of mouse models that faithfully recapitulate human Glioblastoma (GBM) enables genetic and biological dissection of disease initiation and progression and facilitates the systematic evaluation of targeted therapy. A central issue in the accurate generation of such models relates to the possible cellular origins of GBM along the neural stem cell to astrocyte axis. Current mouse models support the view that GBM may originate from the malignant transformation of NSC stem cells, early glial progenitors, and/or mature astrocytes that carry specific combinations of genetic mutations. The development of accurate animal models of GBM is critical in establishing mechanisms underlying tumorigenesis and providing a means for pre-clinical testing of novel therapeutic agents.

The use of transgenic mice for this purpose requires availability of promoter/enhancer elements (or Control Regulatory Modules (CRMs)) that can drive expression of genes in specific cellular compartments. Indeed, a major barrier in brain tumor research is the lack of CRMs that can target subsets of the astrocyte lineage in vivo. For example, the currently available GFAP, nestin and S100b promoters all drive expression in early progenitor cells, which has limited efforts to establish a mechanism of glioma formation by astrocyte de-differentiation in vivo. Thus, in the absence of truly robust astrocyte-specific CRMs, the central question of ‘glioma cell of origin’ in vivo remains ambiguous.

The identification of the astrocyte-specific markers described herein and listed in Tables 2 and 5 has led to the identification of several gene expression domains that may be used to develop new CRMs.

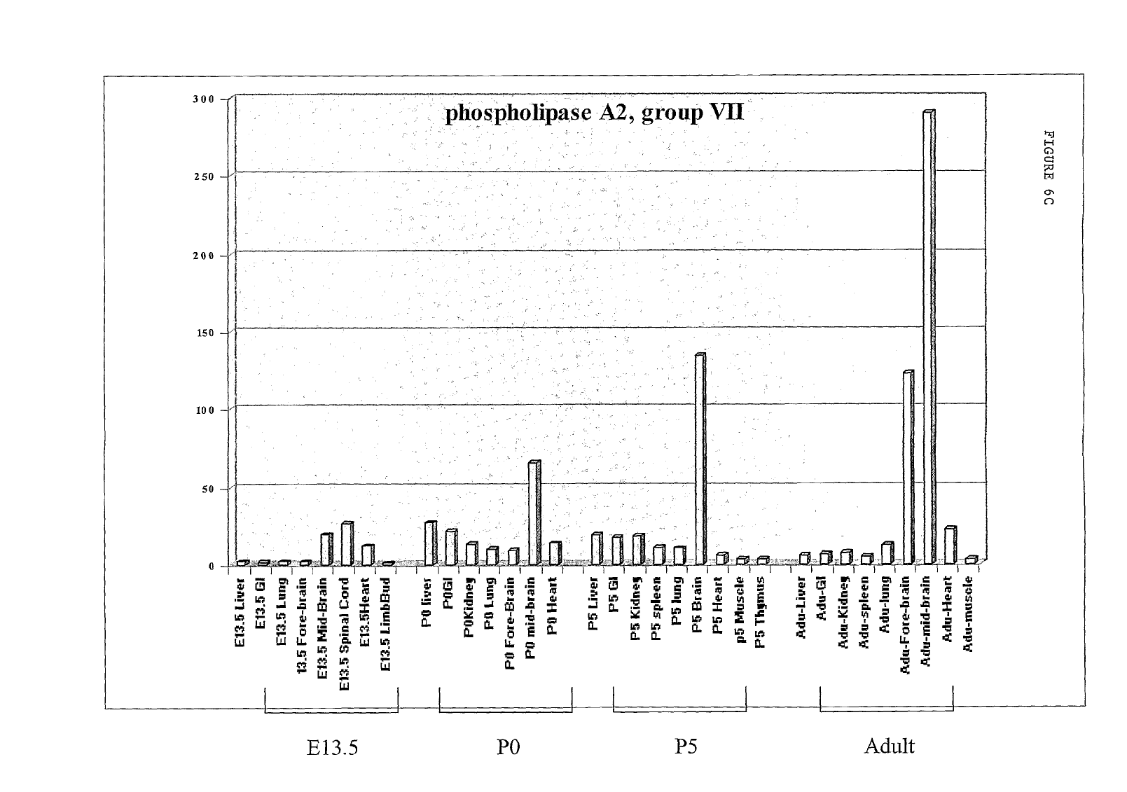

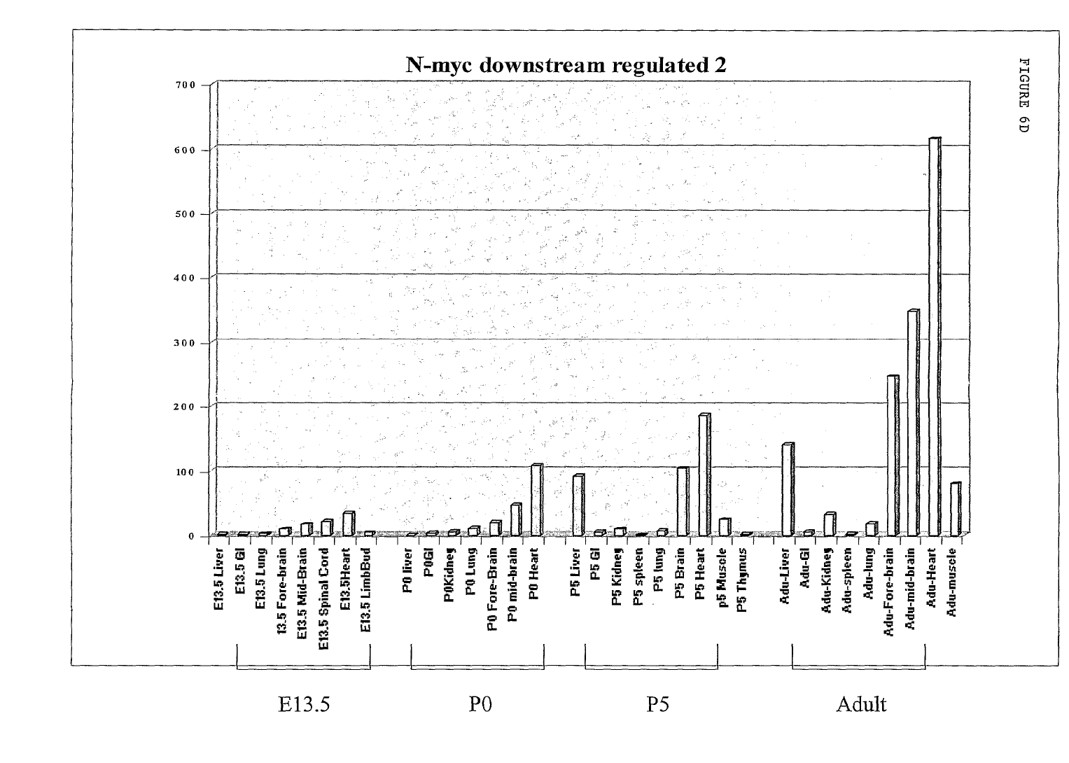

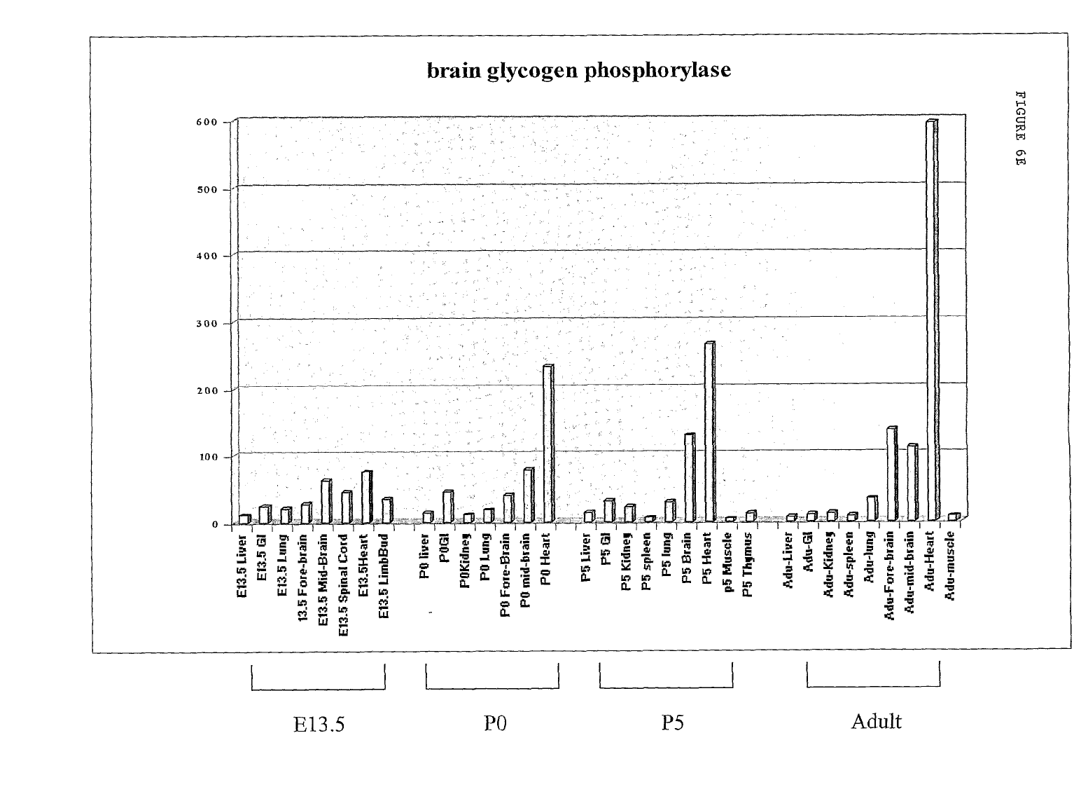

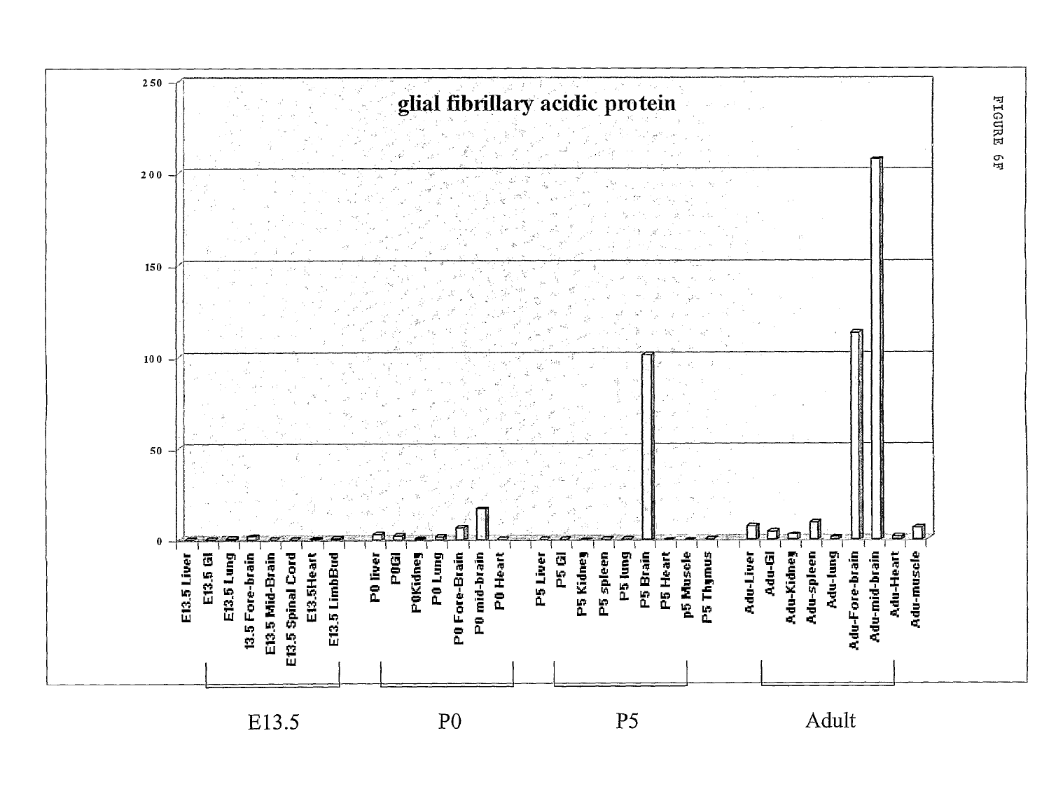

In order to validate the capacity of various CRMs for driving expression of useful reporter genes or Cre recombinase specifically in the mature astrocyte compartment or discrete subsets of such cells, the global expression patterns, of these markers was assessed by quantitative RT-PCR using RNA from the brain and major organs of E13.5, P0, P5 and adult mice. Quantitative PCR was performed using specific primers for the candidate CRMs, compared to a reference control gene (ribosomal RNA gene). As demonstrated in FIGS. 6A-6F, five gene candidates showed high expression in the brain relative to the other organs with increasing expression from P0 to adult.

In order to use these astrocyte-specific CRMs to develop animal models of neurological disease, a targeting plasmid is constructed using genomic DNA fragments derived from Sv129 mouse strain. A neuroglial promoter element (astrocyte-specific CRM) is operably linked to, for example, a Cre recombinase and a neomycin/thymidine kinase cassette is introduced into the EGFR locus. Embryonic stem (ES) cell (derived from Sv129 strain) electroporation, selection and screening are performed using standard gene targeting techniques. Genomic DNA is isolated from neomycin-resistant ES cell clones, digested with BamHI and subjected to hybridization using a probe to detect homologous recombination and the presence of the Cre allele. Chimeric founders are bred with wild-type C57BL/6 mice to obtain offspring containing a germ-line EGFR-Cre allele. These mice are subsequently bred with transgenic mice carrying loxP cites to excise Cre and put EGFR under the control of the neuroglial specific promoter.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of assessing whether a subject is afflicted with a neurological disease, disorder or condition, the method comprising comparing:
a) the amount and/or activity of at least one marker in a subject sample, wherein the at least one marker is selected from the group consisting of the markers listed in Table 2, and
b) the normal amount and/or activity of at the least one marker in a control sample from a subject not afflicted with a neurological disease, disorder or condition,
wherein modulation of the amount and/or activity of the at least one marker in the subject sample compared to the normal amount and/or activity is an indication that the subject is afflicted with a neurological disease, disorder or condition.

2. The method of claim 1, wherein the amount or activity of at least one marker is compared.

3. (canceled)

4. The method of claim 2, wherein the amount of at least one marker is determined by determining the level of expression or by determining the copy number of the marker.

5. (canceled)

6. The method of claim 4, wherein the level of expression of the at least one marker is assessed by detecting the presence in the sample of a protein corresponding to the marker.

7. The method of claim 6, wherein the presence of the protein is detected using a reagent which specifically binds the protein selected from the group consisting of an antibody, an antibody derivative, and an antibody fragment.

8. (canceled)

9. The method of claim 4, wherein the level of expression of the at least one marker in the sample is assessed by detecting the presence of a transcribed polynucleotide or portion thereof, wherein the transcribed polynucleotide comprises the marker.

10. The method of claim 9, wherein the transcribed polynucleotide is an mRNA or a cDNA.

11. (canceled)

12. The method of claim 9, wherein the step of detecting further comprises amplifying the transcribed polynucleotide.

13. The method of claim 4, wherein the level of expression of the at least one marker in the sample is assessed by detecting the presence in the sample of a transcribed polynucleotide which anneals with the marker or anneals with a portion of a polynucleotide wherein the polynucleotide comprises the at least one marker, under stringent hybridization conditions.

14. The method of claim 1, wherein the subject sample is selected from the group consisting of neuroglial tissue, whole blood, serum, plasma, buccal scrape, saliva, cerebrospinal fluid, spinal fluid, urine and stool.

15. The method of claim 1, wherein the at least one marker is selected from the subset of markers in listed in Table 5 or Table 7.

16-21. (canceled)

22. A method of selecting a composition capable of modulating a symptom of neurological disease, disorder or condition, the method comprising:
a) providing a sample comprising astrocytes;
b) contacting said sample with a test compound; and
c) determining the ability of the test compound to modulate the amount and/or activity of at least one marker, wherein the marker is selected from the group consisting of the markers listed in Table 2; and
thereby identifying a composition capable of modulating a symptom of a neurological disease, disorder or condition.

23. The method of claim 22, wherein the astrocytes are isolated from an animal model of a neurological disease, disorder or condition.

24. The method of claim 22, wherein the astrocytes are isolated from a neural cell line.

25. The method of claim 22, wherein the astrocytes are isolated from a subject suffering from a neurological disease, disorder or condition.

26. The method of claim 22, further comprising administering the test compound to an animal model of a neurological disease, disorder or condition.

27. The method of claim 22, wherein the at least one marker is selected from the subset of markers listed in Table 5 or Table 7.

28. A method of treating a subject afflicted with a neurological disease, disorder or condition comprising administering to the subject a therapeutically effective amount of a compound which modulates the amount and/or activity of a gene or protein corresponding to at least one marker listed in Table 2, thereby treating a subject afflicted with a neurological disease, disorder or condition.

29. The method of claim 28, wherein the at least one marker is selected from the subset of markers listed in Table 5 or Table 7.

30-44. (canceled)

45. A kit for assessing whether a subject is afflicted with a neurological disease, disorder or condition, the kit comprising reagents for assessing the amount and/or activity of at least one marker selected from the group consisting of the markers listed in Table 2.

46. The kit of claim 45, wherein the at least one marker is selected from the subset of markers listed in Table 5 or Table 7.

47-53. (canceled)

54. A recombinant vector comprising an astrocyte-specific promoter operably linked to a Cre recombinase.

55. The recombinant vector of claim 54, wherein said vector further comprises an inducible fusion protein.

56-57. (canceled)

58. A cell or cell line comprising the recombinant vector of claim 54.

59. A non-human animal containing the recombinant vector of claim 54.

60-72. (canceled)
